(19) United States
(12) Patent Application Publication
(10) Pub. No.: US 2009/0075949 A1
(43) Pub. Date: Mar. 19, 2009

(54) Use of dipyridamole in combination with antithrombotics for treatment and prevention of thromboembolic diseases

(76) Inventor: Wolfgang Eisert, Hannover (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 11/577,720

(22) PCT Filed: Oct. 21, 2005
(86) PCT No.: PCT/EP05/55446

§ 371 Date: Apr. 22, 2007

(30) Foreign Application Priority Data
Oct. 25, 2004 (EP) 04025283.5

(51) Int. Cl. A61K 31/519; A61K 31/5377; A61K 31/496; A61P 9/00; A61K 31/60

(52) U.S. Cl. 514/161; 514/262.1; 514/236.8; 514/253.09

(57) ABSTRACT

The invention relates to a method of treating and preventing thromboembolic disorders, comprising administering dipyridamole in combination with an antithrombotic selected from direct thrombin inhibitors, factor Xa inhibitors and combined thrombin/factor Xa inhibitors to a patient, pharmaceutical compositions suitable for this method of treatment as well as the use of dipyridamole for the manufacture of these pharmaceutical compositions.

### FIELD OF THE INVENTION

This invention relates to a method of treating and preventing thromboembolic disorders, said method comprising administering a therapeutically effective amount of dipyridamole (DIP) in combination with a therapeutically effective amount of an antithrombotic selected from direct thrombin inhibitors, factor Xa inhibitors and combined thrombin/factor Xa inhibitors to a patient in need thereof. The invention further relates to corresponding pharmaceutical compositions comprising DIP together with an antithrombotic selected from thrombin inhibitors and factor Xa inhibitors, the manufacture thereof, as well as the use of DIP in combination with an antithrombotic selected from thrombin inhibitors and factor Xa inhibitors for the manufacture of a pharmaceutical composition for treatment or prevention of thromboembolic diseases.

### BACKGROUND OF THE INVENTION

Dipyridamole {2,6-bis(diethanolamino)-4,8-dipiperidino-pyrimido[5,4-d]pyrimidine}, closely related substituted pyrimido-pyrimidines and their preparation have been described in e.g. U.S. Pat. No. 3,031,450. Further related substituted pyrimido-pyrimidines and their preparation have been described in e.g. GB 1,051,218, inter alia the compound mopidamol {2,6-bis(diethanolamino)-4-piperidinopyri-mido[5,4-d]pyrimidine}. Dipyridamole was introduced as a coronary vasodilator in the early 1960s. It is also well known having platelet aggregation inhibitor properties due to the inhibition of adenosine uptake. Subsequently, dipyridamole was shown to reduce thrombus formation in a study of arterial circulation of the brain in a rabbit model. These investigations led to its use as an antithrombotic agent; it soon became the therapy of choice for such applications as stroke prevention, maintaining the patency of coronary bypass and valve-replacement, as well as for treatment prior to coronary angioplasty.

Furthermore, the European Stroke Prevention Study 2 (ESPS-2; J Neurol Sci. 1996; 143: 1-13; Neurology 1998; 51: 17-19) proved that treatment by dipyridamole alone was as effective as low-dose aspirin in the reduction of stroke risk, and combination therapy with dipyridamole and aspirin was more than twice as effective as aspirin alone.

Dipyridamole appears to inhibit thrombosis through multiple mechanisms. Early studies showed that it inhibits the uptake of adenosine, which was found to be a potent endogenous anti-thrombotic compound. Dipyridamole was also shown to inhibit cyclic AMP phosphodiesterase, thereby increasing intracellular c-AMP.

By laboratory models reflecting the complex physiology of the blood vessel it could be shown that the vasculature is not a passive conduit, but interacts profoundly with the blood through an intricate system of checks and balances to protect its integrity after vascular accident. Therefore the endothelium produces prostacyclin, a potent inhibitor of aggregation. The normal endothelium is not thrombogenic and prevents the attachment of platelets. Various stimulants precipitate the release of endothelium-derived relaxing factor (EDRF), which inhibits platelet adhesion and aggregation. At the same time, intracellular increase in cGMP was shown to be responsible for relaxation of smooth muscle cells following administration of nitro compounds. Thus the endothelium can inhibit thrombus formation by two separate mechanisms, one mediated by prostacyclin and c-AMP, and the other by EDRF and c-GMP. Dipyridamole appears to enhance both of these antithrombotic mechanisms of the vessel wall, in addition to its adenosine-sparing effects. It stimulates prostacyclin production by increasing intracellular levels of cAMP, and it enhances the strongly anti-thrombotic nitric oxide system by increasing cGMP.

Dipyridamole is highly lipophilic and also has antioxidant properties (Free Radic. Biol. Med. 1995; 18: 239-247) that may contribute to its antithrombotic effect. When oxidized, low density lipoproteins become recognized by the scavenger receptor on macrophages, which is assumed to be the necessary step in the development of atherosclerosis (Ann. Rev. Med. 1992; 43: 219-25).

The inhibition of free radical formation by dipyridamole has been found to inhibit fibrinogenesis in experimental liver fibrosis (Hepatology 1996; 24: 855-864) and to suppress oxygen radicals and proteinuria in experimental animals with amino-nucleoside nephropathy (Eur. J. Clin. Invest. 1998; 28: 877-883; Renal Physiol. 1984; 7: 218-226). Inhibition of lipid peroxidation also has been observed in human nonneoplastic lung tissue (Gen. Pharmacol. 1996; 27: 855-859).

In WO 01/30353 is disclosed that fibrin-dependent microcirculation disorders can be treated by dipyridamole, for example microcirculation disorders caused by metabolic diseases, inflammatory reactions or autoimmune diseases, furthermore peripheral microcirculation disorders or microcirculation disorders associated with increased cell fragmentation.

Furthermore, WO 02/085331 discloses that NO-dependent microcirculation disorders can be treated by dipyridamole, due to the activity as free radical scavenger.

WO 02/34248 discloses a method for increasing tissue perfusion with blood by co-administration of an agent that increases cGMP synthesis and an agent that inhibits cGMP degradation in the cells of the blood vessel walls or in blood cells, e.g. by co-administration of a statin and dipyridamole.

Thrombin is one of the main triggers of thromboembolic disorders. Thrombin is formed within the clotting cascade by clotting factor V and X from its precursor pro-thrombin. Thrombin besides of its fibrin forming capacity activates platelets directly by binding to thrombin receptors on the surface of the platelet as well as other cells relevant to the process of thrombin formation. Thrombin has also been described to react with thrombin receptors on the surface of vessel wall cells, stimulating proliferation and migration of vessel wall cells. So far, direct inhibition of thrombin via synthetic thrombin Inhibitors or indirect inhibition by blocking clotting factor X or any combination of factors in the clotting cascade has been the method of choice to block either thromboembolic events or to block vessel wall thrombosis or restenosis.

Furthermore embolic lodging of tumor metastasis has been connected with clotting activities.

The effects of thrombin are most obvious in areas of slow blood flow such as in low flow venous systems or locally circulating flow such as in vortices behind drastic lumen narrowing or in certain parts of the heart ventricle where wall motion is irregular leading to low or no flow in that part of the atrium or the ventricle. Those conditions are conventionally treated by inhibitors of the clotting cascade or direct thrombin inhibitors or thrombin receptor antagonists, such as unfractionated heparin, low molecular weight heparin, Hirulog or recently developed polyglycans.

It has been hypothesis and tested that after initial trigger of platelet activation such as through arachidonic acid pathway, which is inhibited by ASA or through the binding of ADP to the appropriate ADP receptor on platelet surfaces, subsequent shape change and changes in the outer membrane produces favorable conditions for the binding of the so called pro thrombinase complex. The prothrombinase complex consisting of clothing factor 5A, 10A and prothrombinase bridges by calcium ion to negatively charged phosphor lipids, leading to an acceleration of the formation of thrombin. This acceleration of thrombin formation has been observed by Hemker et al. (Fibrinolysis, International Journal of Fibrinolysis and Thrombolysis, Abstracts of the Eleventh International Congress of Thrombosis: Ljubljana 1990, Volume 4, Supplement 1, abstract No. 182; Thromb Haemost 62 (1), 1989 abstract No. 1211), who described the increase in Km values for thrombin formation to more than 19.000 times, once the prothrombinase complex has been formed and is bound to negatively charged phospholipids on disturbed membranes. It had been hypothesized, that alterations in the outer cell membrane lead to an increased binding of prothrombinase complexes to the surface and thereby to an increase in thrombin formation which is not modulated by inhibition of either ADP receptor blockade or a modulation of the arachidonic acid pathway within the platelet. In early experiments it could be shown, that the binding of the prothrombinase complex to negatively charged phospholipids could be blocked by Annexin V. Annexin V binding to negatively charged phospholipids inhibits the binding of the prothrombinase complex and thereby inhibits the acceleration of thrombin formation on cell surfaces, thrombin itself being the strongest inducer of platelet aggregation.

### SUMMARY OF THE INVENTION

It has now surprisingly been found that DIP in combination with an antithrombotic selected from direct thrombin inhibitors, factor Xa inhibitors and combined thrombin/factor Xa inhibitors advantageously can be used for treatment and/or prevention of thromboembolic and vascular disorders.

DIP is conventionally seen as a platelet inhibitor. However, the combination of platelet inhibitors (such as ASA) with anticoagulant treatment have not shown any significant advantage over anticoagulation treatment alone in the setting of venous thrombosis. The advantages provided by the combined treatment according to the invention may in part be due to the exceptional properties of DIP, which differ significantly from the conventional inhibition of platelet aggregation as seen in all other antiplatelet drugs currently marketed.

In the process of cell apoptosis following metabolic or oxidative stress as well as in the process of activation of platelets, the asymmetry of the outer cell membrane is distorted. By increased binding of Annexin V it has been shown that negatively charged phospholipids get exposed on the outer membrane, which are under physiologic conditions facing the intracellular part of the outer cell membrane. These disturbances in the asymmetry of the outer membrane have also been observed in cells or membrane vesicles after exposure to ionizing radiation. It has been shown in literature, that the coagulation cascade can be significantly accelerated if micro vesicles (membrane particles) are exposed to ionizing radiation, thereby altering the asymmetry of the bilayer and exposing negatively charged phospholipids to the plasma. It is apparent, that membrane disturbance following exposure to oxidative or metabolic stress greatly accelerates the local formation of thrombin and hence fibrin.

Incubation of cells with DIP leads to a significant reduction of Annexin V binding sites compared to pre-incubation in patients with anti-platelet therapy resistance, e.g. ASA or clopidogrel resistance. Reduced formation of Annexin V binding sites reduces excessive formation of thrombin which leads to a insensitivity of platelets to conventional inhibitors of platelet aggregation such as ASA or clopidogrel. As a hypothesis it might be assumed that the antioxidative properties of DIP reduce the impact of oxidative as well as metabolic stress to the outer membrane of cells thereby reducing the formation of Annexin V binding sites. Furthermore, it may be that patients with resistance to ASA or clopidogrel treatment show either a genetic or acquired (e.g. dietary acquired) instability of the asymmetry of the outer cell membrane.

DIP has shown to reduce the binding of Annexin V to the surface of activated platelets. This may be explained by DIP's antioxidant properties and by binding into the outer cell membrane, thereby preventing the local distortion of membrane asymmetry following oxidative or metabolic stress. These stress condition is expected to occur more likely under low flow condition and with significant hypoxia as in the case of slow venous blood flow, explaining the tendency of increased fibrin formation in slow flowing hypoxigenated blood in the venous system. On the other hand even in the arterial tree and under high flow rate, pretreatment with DIP reduces the local formation of fibrin significantly. This inhibition exceeds the level of inhibition by full dose anticoagulant treatment with heparin in experimental animals after angioplasty.

Thus, the stabilization of the outer membrane, hence less exposure of prothrombotic (negatively charged phospholipids) membrane surfaces, provided by DIP significantly enhances the level of inhibition of fibrin formation provided by direct thrombin inhibitors, factor Xa inhibitors and combined thrombin/factor X in the combinations according to the invention.

Viewed from one aspect the present invention provides a method of treatment of the human or non-human animal body, preferably mammalian body, for treating and/or preventing thromboembolic disorders, or medical conditions accompanied or characterized by global or local elevation of thrombin in the plasma or localized elevation of thrombin at a site of low blood flow or other conditions to increase thrombin formation, said method comprising administering a therapeutically effective amount of DIP or a pharmaceutically acceptable salts thereof, in combination with a therapeutically effective amount of an antithrombotic selected from direct thrombin inhibitors, factor Xa inhibitors and combined thrombin/factor Xa inhibitors to a patient in need thereof. The method of the invention can also be combined with the application of a platelet aggregation inhibitor selected from acetylsalicalic acid (ASA), clopidogrel and ticlopidine and the pharmaceutically acceptable salts thereof, ASA being preferred.

Viewed from a further aspect the present invention provides a pharmaceutical composition comprising a therapeutically effective amount of DIP or a pharmaceutically acceptable salt thereof, and a therapeutically effective amount of an antithrombotic selected from direct thrombin inhibitors, factor Xa inhibitors and combined thrombin/factor Xa inhibitors, optionally together with one or more excipients or carriers. In a further embodiment of the invention the pharmaceutical composition comprises additionally a therapeutically effective amount of a platelet aggregation inhibitor selected from ASA, clopidogrel and ticlopidine and the pharmaceutically acceptable salts thereof, ASA being preferred.

Viewed from a still further aspect the present invention provides the use of DIP or a pharmaceutically acceptable salt thereof, in combination with an antithrombotic selected from direct thrombin inhibitors, factor Xa inhibitors and combined thrombin/factor Xa inhibitors, for the manufacture of a pharmaceutical composition for the treatment of the human or non-human animal body, preferably mammalian body, for treating and/or preventing thromboembolic disorders, or medical conditions accompanied or characterized by global- or local elevation of thrombin in the plasma or localized elevation of thrombin at a site of low blood flow or other conditions to increase thrombin formation. In a further embodiment of the invention the pharmaceutical composition to be prepared comprises additionally a therapeutically effective amount of a platelet aggregation inhibitor selected from ASA, clopidogrel and ticlopidine and the pharmaceutically acceptable salts thereof, ASA being preferred.

### DETAILED DESCRIPTION OF THE INVENTION

Thromboembolic disorders which can be treated or prevented according the invention are meant to be such disorders or medical conditions being accompanied or characterized by elevated thrombin formation or thrombin receptor expression or such conditions where elevated thrombin plasma levels or elevated thrombin receptor expression are involved or contribute in pathogenesis or progression of the disorder. This is the case for instance in disorders wherein elevated thrombin activity can lead to increased clot formation, thereby obstructing a venous or an arterial blood vessel at its site or by dislodgement and embolus formation in distant small and large vessels or lead to development of vascular syndromes, damages or diseases, atherosclerotic damages or arthritic conditions or stenosis by thrombin mediated vessel wall alterations such as proliferation and/or migration of vessel wall cells. Elevated thrombin activity are reported in connection with several thrombo-embolic disorders in the scientific literature regarding venous as well as arterial and microcirculatory disorders.

The rationale for the combination with DIP clearly is to achieve a successful treatment or prevention of the indications anticoagulants, e.g. antithrombotics, normally are given for, e.g. the known prevention therapy of cardiovascular risk patients with the aim to reduce the risk for primary or secondary cardiovascular events or ischemic stroke. In general, the underlying basic antithrombotic therapy may be directed to any indication which can be positively influenced by the inhibition of fibrin formation thus improving the blood supply, especially microcircular blood supply, of affected tissues or organs, including but not limited to

The method of treatment according to the invention can be applied for

(a) treatment or prevention of acute myocardial infarction, prevention of myocardial reinfarction,
(b) treatment or prevention of myocardial ischemia (angina pectoris, ischemic heart diseases, chest pain of ischemic etiology), of coronary heart disease or of acute coronary syndromes, secondary prevention of coronary artery disease, treatment and prevention of recurrent ischemic events after acute myocardial infarction, prevention of left ventricular thrombus formation following anterior myocardial infarction,
(c) treatment or prevention of TIA (transient ischemic attacks, or acute cerebrovascular syndromes), of ischemic stroke or prevention of secondary ischemic stroke,
(d) treatment and prevention of complications of (chronic) atrial fibrillation, e.g. stroke prevention in atrial fibrillation,
(d) reduction of the risk for cardiovascular death,
(e) treatment or prevention of ischemic peripheral circulatory disorders, of peripheral vascular disease or of peripheral microcirculation disorders (e.g. Raynaud's disease, tinnitus or sudden loss of hearing),
(f) treatment or prevention of pulmonary hypertension or of pulmonary embolism,
(g) treatment or prevention of thromboembolism, acute treatment and extended secondary prevention of deep vein thrombosis (DVT), prevention of venous thromboembolism after major orthopaedic surgery (e.g. hip or knee replacement),
(h) arterial thrombosis of any vessel, peripheral arterial occlusion, retinal vascular accident, catheter thrombotic occlusion or reocclusion, disseminated intravascular coagulation,
(i) prevention of thromboembolic disorders or complications by endovascular procedures, intra-arterial or intravenous lines, implantation of devices, particularly those exposed to the blood flow, such as stents, prosthetic heart valves, filters, etc, whereby this risk of thrombus formation is reduced by the method of the invention,
(k) prevention of stenosis in vascular grafts, e.g. synthetic vascular grafts, prevention of vascular stent stenosis, prevention of coronary stent stenosis, carotid stent stenosis or peripheral stent stenosis, prevention of stenosis in synthetic grafts used in patients with haemodialysis, prevention of shunt stenosis, prevention of restenosis after angioplasty (e.g. balloon angioplasty, PT(C)A), or preventing reocclusions after bypass operations
in a person in need thereof.

The expression “prevention” used hereinbefore should be understood in the sense that the risk to develop a condition mentioned hereinbefore is reduced, especially in a patient having elevated risk for said conditions, e.g. elevated risk of cardiovascular events or stroke as is the case e.g. in patients who already had a first cardiovascular event, in diabetic, obese and hypertensive patients or heavy smokers. The expression “treatment” means therapeutic treatment of patients having already developed one or more of said conditions in manifest form, including symptomatic treatment in order to relieve symptoms of the specific indication or causal treatment in order to reverse or partially reverse the condition or to delay the progression of the indication as far as this may be possible, depending on the condition and the severity thereof.

The method of the invention is meant as a combination therapy of a patient in need thereof, comprising a basic therapy with an antithrombotic selected from direct thrombin inhibitors, factor Xa inhibitors and combined thrombin/factor Xa inhibitors and a parallel therapy with DIP to improve the efficacy of the basic therapy. The combination therapy is meant to comprise any parallel treatment regimes with the antithrombotic and DIP, wherein either DIP or the antithrombotic may be administered first in a sequential therapy, or both drugs may be administered simultaneously.

The antithrombotics to be used within the method of the invention are all known in the art and comprise direct thrombin inhibitors such as
(1) 1-methyl-2-[N-(4-amidinophenyl)-aminomethyl]-benzimidazol-5-yl-carboxylic acid-N-(2-pyridyl)-N-(2-hydroxycarbonylethyl)-amide, described in WO 98/37075, having the structure

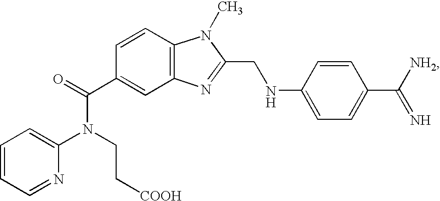

the following prodrug thereof:
(2) dabigatran etexilate (1-methyl-2-[N-[4-(N-n-hexyloxycarbonylamidino)phenyl]-aminomethyl]-benzimidazol-5-yl-carboxylic acid-N-(2-pyridyl)-N-(2-ethoxycarbonyl-ethyl)-amide), also described in WO 98/37075, having the structure

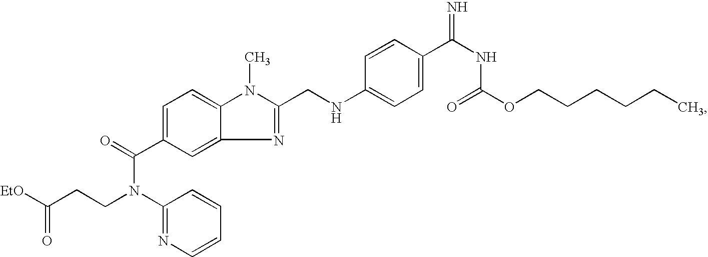

(3) 1-methyl-2-[4-(N-hydroxyamidino)-phenylaminomethyl]-benzimidazol-5-yl-carboxylic acid-(N-2-pyridyl-N-2-ethoxycarbonylethyl)-amide, described in WO 04/014894,
(4) 1-methyl-2-[N-(4-amidinophenyl)-aminomethyl]-benzimidazol-5-yl-carboxylic acid-N-phenyl-N-(2-hydroxycarbonylethyl)-amide (WO 98/37075)
(5) 4-{3-[2,5-dimethyl-4-(N-isopropyl-N-hydroxycarbonylmethylaminocarbonyl-amino)-phenyl]-propargylamino}- benzamidine (DE 199 48 101)
(6) 4-{3-[2,5-dimethyl-4-(N-isopropyl-N-hydroxycarbonylmethylcarbonyl-amino)-phenyl]-propargylamino}-benzamidine (DE 199 48 101)
(7) Melagatran (D. Gustafsson, et al., The Direct Thrombin Inhibitor Melagatran and its Oral Prodrug H 376/95: Intestinal Absorption Properties, Biochemical and Pharmacodynamic Effects, Thromb. Res. 2001, Vol 101(3), 171-181)

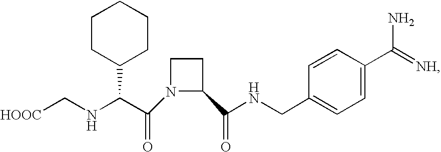

the following orally active prodrug thereof:
(8) Ximelagatran (H-376/95; J. I. Weitz, J. Hirsch; New Anticoagulant Drugs, Chest, 2001, Vol. 119, No. 1 Suppl., 95S-107S)

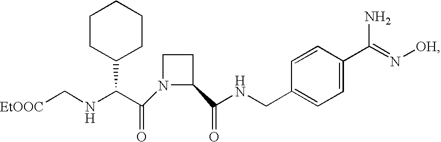

(8a) argatroban,
(8b) bivalrudin,
(8c) SSR-182289A (J. Pharmacol. Exp. Ther. 2003 February; 304(2):567-74), factor Xa inhibitors such as
(9) Razaxaban (DPC-906; Curr Hematol Rep. 2004 September; 3(5): 357-62)
(10) 5-chloro-N-[((5S)-2-oxo-3-[4-(3-oxo-4-morpholinyl)phenyl]-1,3-oxazolidin-5-yl)methyl]-2-thiophencarboxamide (BAY-59-7939, WO 04/60887)
(10a) 1-(4-methoxyphenyl)-7-oxo-6-[4-(2-oxopiperidin-1-yl)phenyl]-4,5,6,7-tetrahydro-1H-pyrazolo[3,4-c]pyridine-3-carboxylic acid amide (INN name: Apixaban),
(11)-(indole-6-carbonyl-D-phenylglycinyl)-4-(1-methyl-piperidin-4-yl)piperazin (LY-517717, WO02/100847)
(12) 2-(5-carbamimidoyl-2-hydroxy-phenyl)-N-[3-methyl-4-(pyrrolidin-1-yl-carbonyl)-phenyl]-acetamide (WO 03/037220)
(13) 2-(3-carbamimidoyl-phenyl)-N-[3-methyl-4-(pyrrolidin-1-yl-carbonyl)-phenyl]-isobutyramide (WO 02/062748)
(14) 2-(5-carbamimidoyl-2-hydroxy-phenyl)-N-[4-(pyrrolidin-1-yl-carbonyl)-3-trifluoromethyl-phenyl]-propionamide (WO 02/062748)
(15) 2-(3-carbamimidoyl-phenyl)-N-[3-bromo-4-(pyrrolidin-1-yl-carbonyl)-phenyl]-3-(pyridin-4-yl)-propionamide (WO 02/062748)
(16) N-(5-carbamimidoyl-2-hydroxy-benzyl)-3-methyl-4-(pyrrolidin-1-yl-carbonyl)-benzamide (WO 02/062778)
(17) ethyl 2-(3-carbamimidoyl-phenyl)-2-[3-methyl-4-(pyrrolidin-1-yl-carbonyl)-benzoylamino]-acetate (WO 02/062778)
(18) (1) N-(5-Amidino-2-hydroxy-benzyl)-3-trifluormethyl-4-(3-aminomethyl-1,4,5,6-tetrahydro-cyclopentapyrazol-1-yl)-benzamide (WO 02/072558)
(19) 6) N-[1-(5-Amidino-2-hydroxy-phenyl)-ethyl]-3-trifluormethyl-4-(4,5,6,7-tetrahydro-benzimidazol-1-yl)-benzamide (WO 02/072558)
(20) N-(5-Amidino-2-hydroxy-benzyl)-3-trifluormethyl-4-(3-methyl-1,4,5,6-tetrahydro-cyclopentapyrazol-1-yl)benzamide (WO 02/072558)
(21) 2-(5-amidino-2-hydroxy-phenyl)-N-[3-trifluoromethyl-4-(pyrrolidin-1-yl-carbonyl)-phenyl]-3-phenyl-propionamide (WO 04/013115)
(22) 4-hydroxy-3-{[6-chloro-7-(pyrrolidin-1-yl-carbonyl)-quinazolin-4-yl]aminomethyl}-benzamidine (WO 2004/080970)
(23) 4-hydroxy-3-{[7-methoxy-6-(pyrrolidin-1-yl-carbonyl)-isoquinolin-1-yl]aminomethyl}-benzamidine (WO 2004/080970)
(24) 4-hydroxy-3-{2-phenyl-1-[7-(pyrrolidin-1-yl-carbonyl)-quinazolin-4-ylamino]-ethyl}-benzamidine (WO 2004/080970)
(25) 4-hydroxy-3-{[6-methyl-7-(pyrrolidin-1-yl-carbonyl)-quinazolin-4-yl]aminomethyl}-benzamidine (WO 2004/080970)
(26) 4-hydroxy-3-{[7-(pyrrolidin-1-yl-carbonyl)-quinazolin-4-yl]aminomethyl}-benzamidine (WO 2004/080970)
(27) ethyl 3-(3-amidino-phenyl)-3-{[6-chloro-7-(pyrrolidin-1-yl-carbonyl)-quinazolin-4-yl]amino}-propionate (WO 2004/080970)
(28) 3-(3-amidino-phenyl)-3-{[6-chloro-7-(pyrrolidin-1-yl-carbonyl)-quinazolin-4-yl]amino}-propionic acid (WO 2004/080970)
(29) N-benzoyl-4-hydroxy-3-{[7-(pyrrolidin-1-yl-carbonyl)-quinazolin-4-yl]aminomethyl}-benzamidine (WO 2004/080970)
(30) N-hydroxy-4-hydroxy-3-{[6-methyl-7-(pyrrolidin-1-yl-carbonyl)-quinazolin-4-yl]aminomethyl}-benzamidine (WO 2004/080970)
(31) N-acetoxymethoxycarbonyl-4-hydroxy-3-{[6-methyl-7-(pyrrolidin-1-yl-carbonyl)-quinazolin-4-yl]aminomethyl}-benzamidine (WO 2004/080970)
   and combined thrombin/factor Xa inhibitors, e.g.
(32) 1-methyl-2-[N-(4-amidinophenyl)-aminomethyl]-5-[N-(hydroxycarbonylmethyl)-quinoline-8-sulphonylamino]-benzimidazole (U.S. Pat. No. 6,121,308)
(33) (R)-2-(4-amidinophenylaminomethyl)-1-methyl-5-[1-(carboxymethylamino)-1-(pyrrolidinocarbonyl)-ethyl]-benzimidazole (WO 00/01704)
(34) 2-(4-amidinophenylaminomethyl)-1-methyl-5-[1-(carboxymethylaminomethyl)-1-(pyrrolidinocarbonyl)-ethyl]-benzimidazole (WO 01/47896)
(35) (R)-2-[4-(N-phenylcarbonylamidino)-phenylaminomethyl]-1-methyl-5-[1-(n-propyloxycarbonylmethylamino)-1-(pyrrolidinocarbonyl)-ethyl]-benzimidazole (WO 01/47896)
(36) 3-{[6-(N-acetyl-N-cyclopentylamino)-7-methyl-isoquinolin-1-yl]aminomethyl}-4-hydroxy-benzamidine (WO 2004/080970)

### (The Following Compounds are Disclosed in WO 2004/056784)

(37) N-[1-(5-chloro-1 H-benzimidazol-2-yl)-ethyl]-3-methyl-4-(2,5-dihydro-pyrrol-1-yl-carbonyl)-benzamide
(38) N-[1-(5-chloro-1H-benzimidazol-2-yl)-ethyl]-3-ethyl-4-(pyrrolidin-1-yl-carbonyl)-benzamide
(39) N-[1-(5-chloro-1H-benzimidazol-2-yl)-ethyl]-3-chloro-4-(2-aminomethyl-pyrrolidin-1-yl-carbonyl)-benzamide
(40) 3-chloro-N-(5-chloro-1H-benzimidazol-2-yl-methyl)-4-(3-oxo-piperazin-1-yl-carbonyl)-benzamide
(41) N-[1-(5-bromo-1H-benzimidazol-2-yl)-ethyl]-3-methyl-4-(pyrrolidin-1-yl-carbonyl)-benzamide
(42) N-[(5-chloro-1H-benzimidazol-2-yl)-phenyl-methyl]-3-methyl-4-(pyrrolidin-1-yl-carbonyl benzamide
(43) N-[1-(5-chloro-1H-benzimidazol-2-yl)-3-methyl-butyl]-3-methyl-4-(pyrrolidin-1-yl-carbonyl)-benzamide
(44) (S)-N-[1-(5-chloro-1H-benzimidazol-2-yl)]ethyl-3-methyl-4-(pyrrolidin-1-yl-carbonyl)-benzamide
(45) N-[(1S)-1-(5-chloro-1H-benzimidazol-2-yl)-ethyl]-3-chloro-4-[(2R/S)-2-aminomethyl-pyrrolidin-1-yl-carbonyl)-benzamide
(46) N-[(1S)-1-(5-chloro-1H-benzimidazol-2-yl)-3-methylsulphanyl-propyl]-3-chloro-4-[(2S)-2-(N-tert.-butoxycarbonyl-aminomethyl)-pyrrolidin-1-yl-carbonyl]-benzamide
(47) N-[(1S)-1-(5-chloro-1H-benzimidazol-2-yl)-butyl]-3-chloro-4-[(2S)-2-aminomethyl-pyrrolidin-1-yl-carbonyl]-benzamide
(48) N-[(1S)-1-(5-chloro-1H-benzimidazol-2-yl)-3-methylsulphanyl-propyl]-3-chloro-4-[(2S)-2-aminomethyl-pyrrolidin-1-yl-carbonyl]-benzamide
(49) N-[(1S)-1-(5-chloro-1H-benzimidazol-2-yl)-3-methylsulphinyl-propyl]-3-chloro-4-[(2S)-2-aminomethyl-pyrrolidin-1-yl-carbonyl]-benzamide
(50) N-[(1S)-1-(5-chloro-1H-benzimidazol-2-yl)-3-methylsulphonyl-propyl]-3-chloro-4-[(2S)-2-aminomethyl-pyrrolidin-1-yl-carbonyl]-benzamide
(51) N-[(1S)-5-(benzyloxycarbonylamino)-1-(5-chloro-1H-benzimidazol-2-yl)-pentyl]-3-methyl-4-(pyrrolidin-1-yl-carbonyl)-benzamide
(52) N-[(1S)-1-(5-chloro-1H-benzimidazol-2-yl)-3-phenyl-propyl]-3-methyl-4-(pyrrolidin-1-yl-carbonyl)-benzamide
(53) N-[(1S)-1-(5-chloro-1H-benzimidazol-2-yl)-3-methylsulphanyl-propyl]-3-methyl-4-(pyrrolidin-1-yl-carbonyl)-benzamide
(54) N-[(1S)-3-benzyloxycarbonyl-1-(5-chloro-1H-benzimidazol-2-yl)-propyl]-3-methyl-4-(pyrrolidin-1-yl-carbonyl)-benzamide
(55) N-[(1S)-1-(5-chloro-1H-benzimidazol-2-yl)-3-(pyrrolidin-1-yl-carbonyl)-propyl]-3-methyl-4-(pyrrolidin-1-yl-carbonyl)-benzamide
(56) N-[(1R)-1-(5-chloro-1H-benzimidazol-2-yl)-2-hydroxy-ethyl]-3-methyl-4-(pyrrolidin-1-yl-carbonyl)-benzamide
(57) N-[1-(5-chloro-1H-benzimidazol-2-yl)-2-methoxy-ethyl]-3-methyl-4-(pyrrolidin-1-yl-carbonyl)-benzamide
(58) N-[(1S)-1-(5-chloro-1H-benzimidazol-2-yl)-3-methoxy-propyl]-3-methyl-4-(pyrrolidin-1-yl-carbonyl)-benzamide
(59) N-[(1R)-2-(C-tert.butoxycarbonyl-methyloxy)-1-(5-chloro-1H-benzimidazol-2-yl)-ethyl]-3-methyl-4-(pyrrolidin-1-yl-carbonyl)-benzamide
(60) N-[(1S)-1-(5-chloro-1H-benzimidazol-2-yl)-3-methylsulphinyl-propyl]-3-methyl-4-(pyrrolidin-1-yl-carbonyl)-benzamide
(61) N-[(5-chloro-1H-benzimidazol-2-yl)-phenyl-methyl]-3-methyl-4-(pyrrolidin-1-yl-carbonyl)-benzamide
(62) N-[1-(5-chloro-1H-benzimidazol-2-yl)-phenyl-methyl]-4-(2,5-dihydro-pyrrol-1-yl-carbonyl)-3-methyl-benzamide
(63) N-[(1S)-1-(5-chloro-1H-benzimidazol-2-yl)-3-methylsulphonylamino-propyl]-3-methyl-4-(pyrrolidin-1-yl-carbonyl)-benzamide
(64) N-{(1S)-1-(5-chloro-1H-benzimidazol-2-yl)-3-[3-(2-chloro-ethyl)-ureido]-propyl}-3-methyl-4-(pyrrolidin-1-yl-carbonyl)-benzamide
(65) N-[(1S)-1-(5-chloro-1H-benzimidazol-2-yl butyl]-3-methyl-4-(pyrrolidin-1-yl-carbonyl)-benzamide
(66) 3-bromo-N-[(1S)-1-(5-chloro-1H-benzimidazol-2-yl)-3-methylsulphanyl-propyl]-4-(pyrrolidin-1-yl-carbonyl)-benzamide
(67) 3-chloro-N-[(1S)-1-(5-chloro-1H-benzimidazol-2-yl)-3-(methylsulphanyl)-propyl]-4-(pyrrolidin-1-yl-carbonyl)-benzamide
(68) 3-bromo-N-[(1S)-1-(5-chloro-1H-benzimidazol-2-yl)-3-(methylsulphonyl)-propyl]-4-(pyrrolidin-1-yl-carbonyl)-benzamide
(69) 3-bromo-N-[(1S)-1-(5-chloro-1H-benzimidazol-2-yl)-3-methylsulphinyl-propyl]-4-(pyrrolidin-1-yl-carbonyl)-benzamide
(70) 3-chloro-N-[(1S)-1-(5-chloro-1H-benzimidazol-2-yl)-ethyl]-4-[(2R)-2-(methylsulphonylamino-methyl)-pyrrolidin-1-yl-carbonyl]-benzamide
(71) (1R)-3-bromo-N-[1-(5-chloro-1H-benzimidazol-2-yl)-2-hydroxy-ethyl]-4-(2,5-dihydro-pyrrol-1-yl-carbonyl)-benzamide
(72) (1R)-3-methyl-N-[1-(5-chloro-1H-benzimidazol-2-yl)-2-methoxy-ethyl]-4-(2,5-dihydro-pyrrol-1-yl-carbonyl)-benzamide
(73) (1R)-3-chloro-N-[1-(5-chloro-1H-benzimidazol-2-yl)-2-hydroxy-ethyl]-4-(2,5-dihydro-pyrrol-1-yl-carbonyl)-benzamide
(74) N-{(1S)-1-(5-chloro-1H-benzimidazol-2-yl)-3-[(3R,S)-3-dimethylamino-pyrrolidin-1-yl]-carbonyl-propyl}-3-methyl-4-(pyrrolidin-1-yl-carbonyl)-benzamide
(75) N-{(1S)-1-(5-chloro-1H-benzimidazol-2-yl)-3-[(2R)-2-hydroxymethyl-pyrrolidin-1-yl-carbonyl]-propyl}-3-methyl-4-(pyrrolidin-1-yl-carbonyl)-benzamide
(76) N-{(1S)-1-(5-chloro-1H-benzimidazol-2-yl)-3-[(2S)-2-hydroxymethyl-pyrrolidin-1-yl-carbonyl]-propyl}-3-methyl-4-(pyrrolidin-1-yl-carbonyl)-benzamide
(77) N-[(1S)-1-(5-chloro-1H-benzimidazol-2-yl)-3-(2-methyl-2,6-diaza-spiro[3.4]oct-6-yl-carbonyl)-propyl]-3-methyl-4-(pyrrolidin-1-yl-carbonyl)-benzamide
(78) N-{(1S)-3-[(1R)-2-(aminocarbonyl)-pyrrolidin-1-yl-carbonyl]-1-(5-chloro-1H-benzimidazol-2-yl)-propyl}-3-methyl-4-(pyrrolidin-1-yl-carbonyl)-benzamide
(79) N-{(1S)-1-(5-chloro-1H-benzimidazol-2-yl)-3-[(2R)-2-tert.butoxycarbonyl-aminomethyl-pyrrolidin-1-yl-carbonyl]-propyl}-3-methyl-4-(pyrrolidin-1-yl-carbonyl)-benzamide
(80) N-{(1S)-1-(5-chloro-1H-benzimidazol-2-yl)-3-[(3R,S)-hydroxymethyl-pyrrolidin-1-yl)-carbonyl]-propyl}-3-methyl-4-(pyrrolidin-1-yl-carbonyl)-benzamide
(81) N-[(1S)-1-(5-chloro-1H-benzimidazol-2-yl)-3-(1,1-dioxo-1-thiomorpholine-4-yl-carbonyl]-propyl]-3-methyl-4-(pyrrolidin-1-yl-carbonyl)-benzamide
(82) N-[(1S)-1-(5-chloro-1H-benzimidazol-2-yl)-3-[(4-methyl-3-oxo-piperazin-1-yl-carbonyl)-propyl]-3-methyl-4-(pyrrolidin-1-yl-carbonyl)-benzamide
(83) N-[(1R)-1-(5-chloro-1H-benzimidazol-2-yl)-2-methoxy-ethyl]-3-methyl-4-(pyrrolidin-1-yl-carbonyl)-benzamide
(84) 3-chloro-N-[(1R)-1-(5-chloro-1H-benzimidazol-2-yl)-2-methoxy-ethyl]-4-(2,5-dihydro-pyrrol-1-yl-carbonyl)-benzamide
(85) 3-bromo-N-[(1R)-1-(5-chloro-1H-benzimidazol-2-yl)-2-methoxy-ethyl]-4-(pyrrolidin-1-yl-carbonyl)-benzamide
(86) 3-bromo-N-[(1R)-1-(5-chloro-1H-benzimidazol-2-yl)-2-methoxy-ethyl]-4-(2,5-dihydro-pyrrol-1-yl-carbonyl)-benzamide
(87) 3-methyl-N-[(1R)-1-(5-chloro-1H-benzimidazol-2-yl)-2-hydroxy-ethyl]-4-(2,5-dihydro-pyrrol-1-yl-carbonyl)-benzamide
(88) N-{(1S)-1-(5-chloro-1H-benzimidazol-2-yl)-3-[(2S)-2-aminomethyl-pyrrolidin-1-yl-carbonyl]-propyl}-3-methyl-4-(pyrrolidin-1-yl-carbonyl)-benzamide
(89) N-{(1S)-1-(5-chloro-1H-benzimidazol-2-yl)-3-[(2R)-2-aminomethyl-pyrrolidin-1-yl-carbonyl]-propyl}-3-methyl-4-(pyrrolidin-1-yl-carbonyl)-benzamide
(90) 3-chloro-N-[(1R,S)-1-(5-chloro-1H-benzimidazol-2-yl)-ethyl]-4-[(2R)-2-methoxymethyl-pyrrolidin-1-yl-carbonyl]-benzamide
(91) 3-chloro-N-[1-(5-chloro-1H-benzimidazol-2-yl)-ethyl]-4-(3,4,5,6-tetrahydro-2H-[2,3]-bipyridinyl-1-yl-carbonyl)-benzamide
(92) N-[(1R)-1-(5-chloro-1H-benzimidazol-2-yl)-2-methoxy-ethyl]-4-(pyrrolidin-1-yl-carbonyl)-3-trifluoromethyl-benzamide
(93) N-[(1S)-1,3-bis-(5-chloro-1H-benzimidazol-2-yl)-propyl]-3-methyl-4-(pyrrolidin-1-yl-carbonyl)-benzamide
(94) 3-chloro-N-[(1S)-1-(5-chloro-1H-benzimidazol-2-yl)-ethyl]-4-[(2R/S)-2-dimethylaminomethyl-pyrrolidin-1-yl-carbonyl]-benzamide
(95) N-[(1S)-1-(5-chloro-1H-benzimidazol-2-yl)-3-methanesulphonylamino-propyl]-4-(2,5-dihydro-pyrrol-1-yl-carbonyl)-3-methyl-benzamide
(96) N-[(1S)-1-(5-chloro-1H-benzimidazol-2-yl)-butyl]-4-(2,5-dihydro-pyrrol-1-yl-carbonyl)-3-methyl-benzamide
(97) 3-chloro-N-[(1S)-1-(5-chloro-1H-benzimidazol-2-yl)-butyl]-4-(2,5-dihydro-pyrrol-1-yl-carbonyl)-benzamide
(98) 3-bromo-N-[(1S)-1-(5-chloro-1H-benzimidazol-2-yl)-butyl]-4-(2,5-dihydro-pyrrol-1-yl-carbonyl)-benzamide
(99) 4-(N-acetyl-N-cyclopentyl-amino)-N-[(1S)-1-(5-chloro-1H-benzimidazol-2-yl)-2-methylsulphanyl-ethyl]-3-methyl-benzamide
(100) 3-chloro-N-[(1S)-1-(5-chloro-1H-benzimidazol-2-yl)-ethyl]-4-[(2R)-2-(pyrrolidin-1-yl-methyl)-pyrrolidin-1-yl-carbonyl]-benzamide
(101) 3-bromo-N-[(1R)-1-(5-bromo-1H-benzimidazol-2-yl)-2-methoxy-ethyl]-4-(2,5-dihydro-pyrrol-1-yl-carbonyl)-benzamide
(102) 3-bromo-N-[(1R)-1-(5-chloro-1H-benzimidazol-2-yl)-2-ethoxy-ethyl]-4-(2,5-dihydro-pyrrol-1-yl-carbonyl)-benzamide
(103) N-[(1R)-2-allyloxy-1-(5-chloro-1H-benzimidazol-2-yl)-ethyl]-4-(2,5-dihydro-pyrrol-1-yl-carbonyl)-3-methyl-benzamide
(104) 3-bromo-N-[(1R)-1-(5-chloro-1H-benzimidazol-2-yl)-2-prop-2-ynyloxy-ethyl]-4-(2,5-dihydro-pyrrol-1-yl-carbonyl)-benzamide
(105) N-[(1S)-1-(5-chloro-1H-benzimidazol-2-yl)-3-(1H-tetrazol-5-yl)-propyl]-3-methyl-4-(pyrrolidin-1-yl-carbonyl)-benzamide
(106) N-[(1R)-1-(5-chloro-1H-benzimidazol-2-yl)-2-hydroxy-ethyl]-4-(2,5-dihydro-pyrrol-1-yl-carbonyl)-3-trifluoromethyl-benzamide
(107) 3-chloro-N-[(1R)-1-(5-bromo-1H-benzimidazol-2-yl)-2-hydroxy-ethyl]-4-(2,5-dihydro-pyrrol-1-yl-carbonyl)-benzamide
(108) 3-bromo-N-[(1R)-1-(5-bromo-1H-benzimidazol-2-yl)-2-hydroxy-ethyl]-4-(pyrrolidin-1-yl-carbonyl)-benzamide
(109) 3-methyl-N-[(1R)-1-(5-bromo-1H-benzimidazol-2-yl)-2-hydroxy-ethyl]-4-(pyrrolidin-1-yl-carbonyl)-benzamide

### (The Following Compounds are Disclosed in WO 2004-058743)

(110) 6-chloro-4-[1-(5-chloro-1H-benzimidazol-2-yl)-ethylamino]-7-(2-aminomethyl-pyrrolidin-1-yl-carbonyl)-quinazoline
(111) 6-chloro-4-[1-(S)-(5-chloro-1H-benzimidazol-2-yl)-ethylamino]-7-(2,5-dihydropyrrol-1-yl-carbonyl)-quinazoline
(112) 6-chloro-4-[1-(S)-(5-chloro-1H-benzimidazol-2-yl)-ethylamino]-7-(pyrrolidin-1-yl-carbonyl)-quinazoline
(113) 4-[1-(5-chloro-1H-benzimidazol-2-yl)-3-methylsulphanyl-propylamino]-6-methyl-7-(pyrrolidin-1-yl-carbonyl)-quinoline
(114) 4-[1-(5-chloro-1H-benzimidazol-2-yl)-ethylamino]-6-methyl-7-(pyrrolidin-1-yl-carbonyl)-quinoline
(115) 4-[1-(5-chloro-1H-benzimidazol-2-yl)-ethylamino]-6-methyl-7-(3-oxo-piperazin-1-yl-carbonyl)-quinoline
(116) 4-[(1R/S)-1-(5-chloro-1H-benzimidazol-2-yl)-ethylamino]-6-methyl-7-[(2R)-2-aminomethyl-pyrrolidin-1-yl-carbonyl]-quinoline
(117) 4-[1-(5-chloro-1H-benzimidazol-2-yl)-3-methylsulphanyl-propylamino]-6-methyl-7-(3-oxo-piperazin-1-yl-carbonyl)-quinoline
(118) 4-[1-(5-chloro-1H-benzimidazol-2-yl)-3-methanesulphonyl-propylamino]-6-methyl-7-(pyrrolidin-1-yl-carbonyl)-quinoline
(119) 6-chloro-4-[(1S)-1-(5-chloro-1H-benzimidazol-2-yl)-ethylamino]-7-[(2R)-2-aminomethyl-pyrrolidin-1-yl-carbonyl]-quinazoline
(120) 6-chloro-4-[(1S)-1-(5-chloro-1H-benzimidazol-2-yl)-2-hydroxy-ethylamino]-7-(2,5-dihydropyrrol-1-yl-carbonyl)-quinazoline
(121) 6-chloro-4-[(1S)-1-(5-chloro-1H-benzimidazol-2-yl)-2-hydroxy-ethylamino]-7-[(2R)-2-aminomethyl-pyrrolidin-1-yl-carbonyl]-quinazoline
(122) 6-chloro-4-[1-(5-chloro-1H-benzimidazol-2-yl)-3-hydroxycarbonylpropylamino]-7-(pyrrolidin-1-yl-carbonyl)-quinazoline
(123) 6-chloro-4-[1-(5-chloro-1H-benzimidazol-2-yl)-3-benzyloxycarbonylpropyl-amino]-7-(pyrrolidin-1-yl-carbonyl)-quinazoline
(124) 6-chloro-4-[(1S)-1-(5-chloro-1H-benzimidazol-2-yl)-3-methylsulphanyl-propylamino]-7-[(2R)-2-tert.-butyloxycarbonyl-aminomethyl-pyrrolidin-1-yl-carbonyl]-quinazoline
(125) 6-chloro-4-[(1S)-1-(5-chloro-1H-benzimidazol-2-yl)-3-methylsulphanyl-propylamino]-7-(pyrrolidin-1-yl-carbonyl)-quinazoline
(126) 6-chloro-4-[(1S)-1-(5-chloro-1H-benzimidazol-2-yl)-3-methoxy-propylamino]-7-(2,5-dihydropyrrol-1-yl-carbonyl)-quinazoline
(127) 6-chloro-4-[(1S)-1-(5-chloro-1H-benzimidazol-2-yl)-3-methanesulphonyl-propylamino]-7-(pyrrolidin-1-yl-carbonyl)-quinazoline
(128) 6-chloro-4-[(1S)-1-(5-chloro-1H-benzimidazol-2-yl)-3-methylsulphanyl-propylamino]-7-[(2R)-2-aminomethyl-pyrrolidin-1-yl-carbonyl]-quinazoline
(129) 6-chloro-4-[(1S)-1-(5-chloro-1H-benzimidazol-2-yl)-3-methanesulphinyl-propylamino]-7-(pyrrolidin-1-yl-carbonyl)-quinazoline
(130) 6-chloro-4-[(1S)-1-(5-chloro-1H-benzimidazol-2-yl)-3-benzyloxycarbonyl-propylamino]-7-(pyrrolidin-1-yl-carbonyl)-quinazoline
(131) 6-chloro-4-[(1S)-1-(5-chloro-1H-benzimidazol-2-yl)-2-hydroxy-ethylamino]-7-(piperazin-3-on-1-yl-carbonyl)-quinazoline
(132) 6-chloro-4-[(1S)-1-(5-chloro-1H-benzimidazol-2-yl)-3-hydroxycarbonylpropyl-amino]-7-[(2S)-2-aminomethyl-pyrrolidin-1-yl-carbonyl]-quinazoline
(133) 6-chloro-4-[(1S)-1-(5-chloro-1H-benzimidazol-2-yl)-3-methanesulphonyl-propylamino]-7-[(2R)-2-tert.-butyloxycarbonyl-aminomethyl-pyrrolidin-1-yl-carbonyl]-quinazoline
(134) 6-chloro-4-[(1S)-1-(5-chloro-1H-benzimidazol-2-yl)-3-methanesulphonyl-propylamino]-7-[(2R)-2-aminomethyl-pyrrolidin-1-yl-carbonyl]-quinazoline
(135) 6-chloro-4-[(1S)-1-(5-chloro-1H-benzimidazol-2-yl)-ethylamino]-7-(thiazolidin-3-yl-carbonyl)-quinazoline
(136) 6-chloro-4-[(1S)-1-(5-chloro-1H-benzimidazol-2-yl)-3-ethoxycarbonylpropyl-amino]-7-(pyrrolidin-1-yl-carbonyl)-quinazoline
(137) 4-[(1S)-1-(5-chloro-1H-benzimidazol-2-yl)-ethylamino]-6-methyl-7-(pyrrolidin-1-yl-carbonyl)-quinazoline
(138) 4-[(1S)-1-(5-chloro-1H-benzimidazol-2-yl)-ethylamino]-6-methyl-7-(pyrrolidin-1-yl-carbonyl)-quinazoline
(139) 6-chloro)-4-[(1S)-1-(5-chloro-1H-benzimidazol-2-yl)-3-methanesulphinyl-propylamino]-7-[(2R)-2-aminomethyl-pyrrolidin-1-yl-carbonyl]-quinazoline
(140) 4-[(1S)-1-(5-chloro-1H-benzimidazol-2-yl)-3-methylsulphanyl-propylamino]-6-methyl-7-(2,5-dihydropyrrol-1-yl-carbonyl)-quinazoline
(141) 6-bromo-4-[(1S)-1-(5-chloro-1H-benzimidazol-2-yl]ethylamino]-7-(2,5-dihydropyrrol-1-yl-carbonyl)-quinazoline
(142) 6-chloro-4-[(1S)-1-(5-chloro-1H-benzimidazol-2-yl)-3-ethoxycarbonylpropyl-amino]-7-(2,5-dihydropyrrol-1-yl-carbonyl)-quinazoline
(143) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(144) 6-chloro-4-[(1S)-1-(5-chloro-1H-benzimidazol-2-yl)-butylamino]-7-(2,5-dihydropyrrol-1-yl-carbonyl)-quinazoline
(145) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(146) 6-chloro-4-[(1S)-1-(5-chloro-1H-benzimidazol-2-yl)-2-methoxy-ethylamino]-7-(2,5-dihydropyrrol-1-yl-carbonyl)-quinazoline
(147) 6-chloro-4-[1-(5-chloro-1H-benzimidazol-2-yl)-3-diethylaminocarbonyl-propyl-amino]-7-(pyrrolidin-1-yl-carbonyl)-quinazoline
(148) 6-chloro-4-[1-(5-chloro-1H-benzimidazol-2-yl)-3-[N-methyl-N-piperidin-4-yl-amino]-carbonyl-propyl-amino]-7-(pyrrolidin-1-yl-carbonyl)-quinazoline
(149) 6-chloro-4-[1-(5-chloro-1H-benzimidazol-2-yl)-3-[4-methyl-piperazin-1-yl]-carbonyl-propyl-amino]-7-(pyrrolidin-1-yl-carbonyl)-quinazoline
(150) 6-chloro-4-[1-(5-chloro-1H-benzimidazol-2-yl)-3-(C-piperidin-4-yl-methylamino)-carbonyl-propyl-amino]-7-(pyrrolidin-1-yl-carbonyl)-quinazoline
(151) 6-chloro-4-[1-(5-chloro-1H-benzimidazol-2-yl)-3-(N-benzyl-N-methyl-amino)-carbonyl-propyl-amino]-7-(pyrrolidin-1-yl-carbonyl)-quinazoline
(152) 4-[(1S)-1-(5-chloro-1H-benzimidazol-2-yl)-3-allyloxycarbonylpropyl-amino]-6-methyl-7-(2,5-dihydropyrrol-1-yl-carbonyl)-quinazoline
(153) 6-bromo-4-[(1S)-1-(5-chloro-1H-benzimidazol-2-yl)-3-allyloxycarbonylpropyl-amino]-7-(2,5-dihydropyrrol-1-yl-carbonyl)-quinazoline
(154) 6-chloro-4-[(1S)-1-(5-chloro-1H-benzimidazol-2-yl)-2-methoxy-ethylamino]-7-(pyrrolidin-1-yl-carbonyl)-quinazoline
(155) 6-chloro-4-[(1S)-1-(5-chloro-1H-benzimidazol-2-yl)-3-methanesulphonyl-propylamino]-1-oxy-7-[(2R)-2-aminomethyl-pyrrolidin-1-yl-carbonyl]-quinazoline
(156) 6-chloro-4-[(1S)-1-(5-chloro-1H-benzimidazol-2-yl)-ethylamino]-7-[(2S)-2-(pyrrolidin-1-yl-methyl)-pyrrolidin-1-yl-carbonyl]-quinazoline
(157) 6-chloro-4-[(1S)-1-(5-chloro-1H-benzimidazol-2-yl)-ethylamino]-7-[(2R/S)-2-aminomethyl-thiazolidinyl-carbonyl]-quinazoline
(158) 6-chloro-4-[(1S)-1-(5-chloro-1H-benzimidazol-2-yl)-3-methanesulphonyl-propylamino]-7-[(2R)-2-(methanesulphonyl-aminomethyl)-pyrrolidin-1-yl-carbonyl]-quinazoline
(159) 6-chloro-4-{1-(5-chloro-1H-benzimidazol-2-yl)-3-[(1,2,3,4-tetrahydroiso-quinolin-1-yl)-carbonyl-propyl-amino]}-7-(pyrrolidin-1-yl-carbonyl)-quinazoline
(160) 6-chloro-4-[1-(5-chloro-1H-benzimidazol-2-yl)-3-(benzylamino-carbonyl)-propyl-amino]-7-(pyrrolidin-1-yl-carbonyl)-quinazoline
(161) 6-chloro-4-{1-(5-chloro-1H-benzimidazol-2-yl)-3-[(N-methyl-N-phenethyl-amino-carbonyl)-propyl-amino]}-7-(pyrrolidin-1-yl-carbonyl)-quinazoline
(162) 6-chloro-4-[1-(5-chloro-1H-benzimidazol-2-yl)-3-(hydroxyethylamino-carbonyl)-propyl-amino]-7-(pyrrolidin-1-yl-carbonyl)-quinazoline
(163) 6-chloro-4-{1-(5-chloro-1H-benzimidazol-2-yl)-3-[(C-pyridin-3-yl-methylamino-carbonyl)-propyl-amino]}-7-(pyrrolidin-1-yl-carbonyl)-quinazoline
(164) 6-chloro-4-{1-(5-chloro-1H-benzimidazol-2-yl)-3-[(1-oxa-3,8-diaza-spiro[4.5]decan-2-on-8-yl)-carbonyl]-propyl-amino}-7-(pyrrolidin-1-yl-carbonyl)-quinazoline
(165) 6-chloro-4-[1-(5-chloro-1H-benzimidazol-2-yl)-3-(morpholin-4-yl-carbonyl)-propyl-amino]-7-(pyrrolidin-1-yl-carbonyl)-quinazoline
(166) 6-chloro-4-[1-(5-chloro-1H-benzimidazol-2-yl)-3-(C-cyclohexyl-methylamino-carbonyl)-propyl-amino]-7-(pyrrolidin-1-yl-carbonyl)-quinazoline
(167) 6-chloro-4-[1-(5-chloro-1H-benzimidazol-2-yl)-3-(methoxyethylamino-carbonyl)-propyl-amino]-7-(pyrrolidin-1-yl-carbonyl)-quinazoline
(168) 6-chloro-4-[1-(5-chloro-1H-benzimidazol-2-yl)-3-(dimethylaminoethyl-amino-carbonyl)-propyl-amino]-7-(pyrrolidin-1-yl-carbonyl)-quinazoline
(169) 6-chloro-4-[1-(5-chloro-1H-benzimidazol-2-yl)-3-(cyclopropylamino-carbonyl)-propyl-amino]-7-(pyrrolidin-1-yl-carbonyl)-quinazoline
(170) 6-chloro-4-{(1R/S)-1-(5-chloro-1H-benzimidazol-2-yl)-3-[C-(2R/S)-tetrahydrofuran-2-yl-methylamino-carbonyl)-propyl-amino]}-7-(pyrrolidin-1-yl-carbonyl)-quinazoline
(171) 6-chloro-4-[1-(5-chloro-1H-benzimidazol-2-yl)-3-(dimethylaminopropylamino-carbonyl)-propyl-amino]-7-(pyrrolidin-1-yl-carbonyl)-quinazoline
(172) 6-chloro-4-[1-(5-chloro-1H-benzimidazol-2-yl)-3-(aminoethylamino-carbonyl)-propyl-amino]-7-(pyrrolidin-1-yl-carbonyl)-quinazoline
(173) 6-chloro-4-[1-(5-chloro-1H-benzimidazol-2-yl)-3-(2,2,2-trifluoroethylamino-carbonyl)-propyl-amino]-7-(pyrrolidin-1-yl-carbonyl)-quinazoline
(174) 6-chloro-4-{1-(5-chloro-1H-benzimidazol-2-yl)-3-[N-(2-dimethylamino-ethyl)-N-methyl-amino-carbonyl]-propyl-amino}-7-(pyrrolidin-1-yl-carbonyl)-quinazoline
(175) 6-chloro-4-[1-(5-chloro-1H-benzimidazol-2-yl)-3-(N-piperidin-2-yl-aminocarbonyl)-propyl-amino]-7-(pyrrolidin-1-yl-carbonyl)-quinazoline
(176) 6-chloro-4-{1-(5-chloro-1H-benzimidazol-2-yl)-3-[C-(tetrahydropyran-4-yl)-methylamino-carbonyl]-propyl-amino}-7-(pyrrolidin-1-yl-carbonyl)-quinazoline
(177) 6-chloro-4-[1-(5-chloro-1H-benzimidazol-2-yl)-3-(4-hydroxypiperidin-1-yl-carbonyl)-propyl-amino]-7-(pyrrolidin-1-yl-carbonyl)-quinazoline
(178) 6-chloro-4-{1-(5-chloro-1H-benzimidazol-2-yl)-3-[C-(pyridin-4-yl)-methylamino-carbonyl]-propyl-amino}-7-(pyrrolidin-1-yl-carbonyl)-quinazoline
(179) 6-chloro-4-[1-(5-chloro-1H-benzimidazol-2-yl)-3-(N-methylaminocarbonylmethyl-N-methyl-amino-carbonyl)-propyl-amino]-7-(pyrrolidin-1-yl-carbonyl)-quinazoline
(180) 6-chloro-4-{1-(5-chloro-1H-benzimidazol-2-yl)-3-[N-(2-(1H)-imidazol-4-yl)-ethyl)-N-methyl-amino-carbonyl]-propyl-amino}-7-(pyrrolidin-1-yl-carbonyl)-quinazoline
(181) 6-chloro-4-[1-(5-chloro-1H-benzimidazol-2-yl)-3-(1-thiazolidin-3-yl-carbonyl)-propyl-amino]-7-(pyrrolidin-1-yl-carbonyl)-quinazoline
(182) 6-chloro-4-[1-(5-chloro-1H-benzimidazol-2-yl)-3-(N-cyclopropyl-N-methyl-amino-carbonyl propyl-amino]-7-(pyrrolidin-1-yl-carbonyl)-quinazoline
(183) 6-chloro-4-[1-(5-chloro-1H-benzimidazol-2-yl)-3-(N-cyclopropylmethyl-N-methyl-amino-carbonyl)-propyl-amino]-7-(pyrrolidin-1-yl-carbonyl)-quinazoline
(184) 6-chloro-4-[1-(5-chloro-1H-benzimidazol-2-yl)-3-(cyclopentylamino-carbonyl)-propyl-amino]-7-(pyrrolidin-1-yl-carbonyl)-quinazoline
(185) 6-chloro-4-[1-(5-chloro-1H-benzimidazol-2-yl)-3-(N-piperidin-4-yl-aminocarbonyl)-propyl-amino]-7-(pyrrolidin-1-yl-carbonyl)-quinazoline
(186) 6-chloro-4-{1-(5-chloro-1H-benzimidazol-2-yl)-3-[C-(pyridin-2-yl)-methylamino-carbonyl]-propyl-amino}-7-(pyrrolidin-1-yl-carbonyl)-quinazoline
(187) 6-chloro-4-[(1S)-1-(5-chloro-1H-benzimidazol-2-yl)-3-hydroxycarbonyl-propylamino]-7-(pyrrolidin-1-yl-carbonyl)-quinazoline
(188) 6-chloro-4-[(1S)-1-(5-chloro-1H-benzimidazol-2-yl)-ethylamino]-7-(5,6,7,8-tetrahydro-[1,2,4]triazolo[4,3a]pyridin-4-yl)-quinazoline
(189) 6-chloro-4-[(1S)-1-(5-chloro-1H-benzimidazol-2-yl)-3-(1,1-dioxo-isothiazolidin-2-yl)-propyl-amino]-7-(2,5-dihydropyrrol-1-yl-carbonyl)-quinazoline
(190) 6-chloro-4-[(1S)-1-(5-chloro-1H-benzimidazol-2-yl)-3-methanesulphonylamino-propyl-amino]-7-(2,5-dihydropyrrol-1-yl-carbonyl)-quinazoline
(191) 4-[(1 S)-1-(5-chloro-1H-benzimidazol-2-yl)-3-(methylsulphanyl)-propylamino]-6-methoxy-7-(2,5-dihydropyrrol-1-yl-carbonyl quinazoline
(192) 4-[(1S)-1-(5-chloro-1H-benzimidazol-2-yl)-2-methoxy-ethylamino]-6-methoxy-7-(2,5-dihydropyrrol-1-yl-carbonyl)-quinazoline
(193) 6-chloro-4-[(1S)-1-(5-chloro-1H-benzimidazol-2-yl)-2-methoxy-ethylamino]-7-(thiazolidinyl-carbonyl)-quinazoline
(194) 4-[(1S)-1-(5-chloro-1H-benzimidazol-2-yl)-2-methoxy-ethylamino]-6-methyl-7-(2,5-dihydropyrrol-1-yl-carbonyl)-quinazoline
(195) 4-[(1S)-1-(5-chloro-1H-benzimidazol-2-yl)-2-methoxy-ethylamino]-6-methyl-7-(thiazolidinyl-carbonyl)-quinazoline
(196) 6-bromo-4-[(1S)-1-(5-chloro-1H-benzimidazol-2-yl)-2-methoxy-ethylamino]-7-(2,5-dihydropyrrol-1-yl-carbonyl)-quinazoline
(197) 6-bromo-4-[(1S)-1-(5-chloro-1H-benzimidazol-2-yl)-2-methoxy-ethylamino]-7-(thiazolidinyl-carbonyl)-quinazoline
(198) 6-chloro-4-[(1S)-1-(5-chloro-1H-benzimidazol-2-yl)-ethylamino]-7-(6,7,8,9-tetrahydro-[1,2,4]triazolo[4,3-a]pyridin-4-yl)-quinazoline
(199) 6-chloro-4-{1-(5-chloro-1H-benzimidazol-2-yl)-3-[2-(pyridin-4-yl-amino)-ethylamino-carbonyl]-propylamino}-7-(pyrrolidin-1-yl-carbonyl)-quinazoline
(200) 4-[(1S)-1-(5-bromo-1H-benzimidazol-2-yl)-2-methoxy-ethylamino]-6-chloro-7-(2,5-dihydropyrrolyl-carbonyl)-quinazoline and
(201) 4-[(1S)-1-(5-bromo-1H-benzimidazol-2-yl)-ethylamino]-6-chloro-7-(2,5-dihydropyrrolyl-carbonyl)-quinazoline,
   their stereoisomers such as enantiomers and diastereomers, mixtures of stereoisomers such as racemates, prodrugs, pharmacologically acceptable salts, solvates, e.g. hydrates, and physical modifications thereof, e.g. polymorphs.

Prodrugs of the drugs mentioned above are such derivatives containing one or more groups capable of being cleaved in vivo, particularly a group which can be converted in-vivo into a carboxy group or/and a group capable of being cleaved in vivo from an imino or amino group. Compounds containing two groups capable of being cleaved in vivo are so-called double prodrugs. Groups which can be converted in-vivo into a carboxy group and groups capable of being cleaved in vivo from an imino or amino group are disclosed e.g. in WO 98/37075, being herewith incorporated by reference, as well as in other WO publications cited hereinbefore in connection with specific antithrombotics.

According to the method of treatment and/or prevention according to the invention it is of advantage to maintain a plasma level of dipyridamole of about 0.2 to 5 μg/L, preferably of about 0.4 to 5 μg/L, especially of about 0.5 to 2 μg/L or particularly of about 0.8 to 1.5 μg/L. This can be achieved using any of the oral dipyridamole retard, instant or the parenteral formulations on the market, the retard formulations being preferred, for instance those available under the trademark Persantin®, or, for the combination therapy with low-dose ASA, using those formulations available under the trademark Asasantin® or Aggrenox®: Dipyridamol retard formulations are also disclosed in EP-A-0032562, instant formulations are disclosed in EP-A-0068191 and combinations of ASA with dipyridamole are disclosed in EP-A-0257344 which are incorporated by reference. In case of mopidamole also oral retard, instant or a parenteral formulations can be used, e.g. those disclosed in GB 1,051,218 or EP-A-0,108,898 which are incorporated by reference, retard formulations being preferred.

Dipyridamole can be administered orally in a daily dosage of 25 to 1000 mg, preferably 50 to 900 mg, more preferred 100 to 480 mg, most preferred 150 to 400 mg. For long-term treatment it is of advantage to administer repeated doses such as a dose of 50 to 500 mg, preferably 200 to 400 mg of dipyridamole retard or any other instant release formulation three or four times a day. For parenteral administration dipyridamole could be given in a dosage of 0.5 to 5 mg/kg body weight, preferably 1 to 3.5 mg/kg body weight, during 24 hours as slow i.v. infusion (not faster than 0.2 mg/min).

The antithrombotic can be administered either in accordance with its usual dosage range or, preferably, with a dose below its usual dosage range. This could be a dose where the antithrombotic, when given alone, does not effectively prevent fibrin formation, but in combination with DIP treatment according to the invention does. The dosage for the antithrombotic in combination with DIP is appropriately 1/50 of the lowest dose normally recommended up to 1/1 of the normally recommended dosage, e.g. 1/20 to ½ and preferably 1/10 to ½, preferably ⅕ to ½. The normally recommended dose for the antithrombotic drug is as follows:

intravenously, preferably administered slowly, or subcutaneously: 0.001 to 3.0 mg/kg body weight (bw) or, preferably, 0.005 to 0.5 mg/kg bw or, more preferred, 0.01 to 0.1 mg/kg bw, once or two times a day, and
orally: 0.03 to 30 mg/kg bw or, preferably, 0.1 to 10 mg/kg bw or, more preferred, 0.1 to 1 mg/kg bw, one to four times a day.

For instance, the normally recommended dose for the antithrombotics (1) to (201) drug may be the dose disclosed in Rote Liste® 2004, Editio Cantor Verlag Aulendorf, Germany, or to Physician's Desk Reference, 58 edition, 2004, e.g. exemplary for melagatran 3 mg/0.3 ml s.c. two times a day, or for ximelagatran 24 mg orally two times a day,

for Apixaban 2, 5-125 mg orally one or two times a day,
the normally recommended dose for (8a) argatroban may be 0.1 to 2.8 μg/kg/min, e.g. about 0.2, 0.5, 1.0, 1.5, 2.0, 2.5 μg/kg/min iV (compare Ann. Pharmacother. 2005, Aug. 30),
the normally recommended dose for (8b) bivalrudin may be 0.1 to 2.8 μg/kg/min, preferably 1.75-2.2 mg/kg/h, e.g. about 0.2, 0.5, 1.0, 1.5, 2.0, 2.5 μg/kg/min iV (compare Annals of Thoracic Surgery 2004, 77 (925-931)).

### Formulations and Dosages: Opional Platelet Aggregation Inhibitors/ASA

With respect to ASA any of the oral formulations on the market may be used. Reference is made to Rote Liste® 2004, Editio Cantor Verlag Aulendorf, Germany, or to Physician's Desk Reference, 58 edition, 2004. This component of the medication may be administered orally in a daily dosage of 10 to 1000 mg, preferably 25 to 400 mg, e.g. 100 to 300 mg, most preferred 30 to 75 mg, for instance 25 mg twice a day.

### Formulations and Dosages: Opional Platelet Aggregation Inhibitors/Clopidogrel

Suitable oral formulations of clopidogrel are disclosed in Rote Liste® 2004, Editio Cantor Verlag Aulendorf, Germany, or in Physician's Desk Reference, 58 edition, 2004, and may contain from 25 mg to 500 mg, preferably from 75 mg to 375 mg, and most preferably from 75 mg to 150 mg of clopidogrel. For example, the formulation used may contain 25 mg, 50 mg, 75 mg, 150 mg, 250 mg, or 500 mg of clopidogrel. Oral administration may be in one or divided doses of two, three, or four times daily. A single daily dose is preferred. Clopidogrel is on the market under the brand names Plavix® and Iscover®.

### Formulations and Dosages: Opional Platelet Aggregation Inhibitors/Ticlopidine

Suitable oral formulations of ticlopidine are disclosed in Rote Liste® 2004, Editio Cantor Verlag Aulendorf, Germany, or in Physician's Desk Reference, 58 edition, 2004, and may contain from 25 mg to 600 mg, preferably from 100 mg to 400 mg, and most preferably from 200 mg to 300 mg of ticlopidine. For example, the formulation may contain 25 mg, 50 mg, 75 mg, 150 mg, 250 mg, or 500 mg of ticlopidine. Oral administration may be in one or divided doses of two, three, or four times daily. A single daily dose is preferred.

The pharmaceutical compositions according to the invention are meant to comprise dipyridamole or a pharmaceutically acceptable salt thereof, together with an antithrombotic selected from the compounds (1) to (201) mentioned hereinbefore, and may optionally comprise one or more excipients or auxiliary compounds. In a further embodiment the pharmaceutical combinations may comprise additionally a platelet aggregation inhibitor selected from acetylsalicalic acid (ASA), clopidogrel and ticlopidine and the pharmaceutically acceptable salts thereof, ASA being preferred. The pharmaceutical compositions preferably are adapted for administration of the dosages mentioned hereinbefore.

The pharmaceutical compositions according to the invention are meant to comprise a fixed dose combination comprising the active ingredients in one formulation together as well as a kit of parts comprising the active ingredients each in a separate containment, preferably in one package. The pharmaceutical composition may be adapted for simultaneous, separate or sequential administration.

For instance, a pharmaceutical composition according to the invention comprises dipyridamole or a pharmaceutically acceptable salt thereof, and a second active ingredient selected from compounds (1) to (201) and the physiologically acceptable salts thereof, optionally together with one or more excipients or carriers.

In a further embodiment, the pharmaceutical composition according to the invention comprises dipyridamole or a pharmaceutically acceptable salt thereof, a second active ingredient selected from compounds (1) to (201) and the physiologically acceptable salts thereof, a platelet aggregation inhibitor selected from acetylsalicalic acid (ASA), clopidogrel and ticlopidine and the pharmaceutically acceptable salts thereof, optionally together with one or more excipients or carriers.

For preparation of a pharmaceutical composition according to the invention the active ingredients may be formulated, with one or more inert conventional carriers and/or diluents, e.g. with corn starch, lactose, glucose, mannitol, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

Suitable formulations of compounds (1) to (201) are described in the prior art, e.g the references cited in the list of compounds hereinbefore.

1. A method of treating and/or preventing an indication selected from thromboembolic disorders, said method comprising administering a therapeutically effective amount of dipyridamole (DIP) in combination with a therapeutically effective amount of an antithrombotic selected from direct thrombin inhibitors, factor Xa inhibitors and combined thrombin/factor Xa inhibitors to a patient in need thereof.

2. The method of claim 1, wherein the antithrombotic is selected from the group consisting of
(1) 1-methyl-2-[N-(4-amidinophenyl)-aminomethyl]-benzimidazol-5-yl-carboxylic acid-N-(2-pyridyl)-N-(2-hydroxycarbonylethyl)-amide,
(2) dabigatran etexilate (1-methyl-2-[N-[4-(N-n-hexyloxycarbonylamidino)phenyl]-aminomethyl]-benzimidazol-5-yl-carboxylic acid-N-(2-pyridyl)-N-(2-ethoxycarbonyl-ethyl)-amide);
(3) 1-methyl-2-[4-(N-hydroxyamidino)-phenylaminomethyl]-benzimidazol-5-yl-carboxylic acid-(N-2-pyridyl-N2-ethoxycarbonylethyl)-amide
(4) 1-methyl-2-[N-(4-amidinophenyl)-aminomethyl]-benzimidazol-5-yl-carboxylic acid-N-phenyl-N-(2-hydroxycarbonylethyl)-amide
(5) 4-{3-[2,5-dimethyl-4-(N-isopropyl-N-hydroxycarbonylmethylaminocarbonyl-amino)-phenyl]-propargylamino}-benzamidine
(6) 4-{3-[2,5-dimethyl-4-(N-isopropyl-N-hydroxycarbonylmethylcarbonyl-amino)-phenyl]-propargylamino}-benzamidine
(7) Melagatran
(8) Ximelagatran
(8a) argatroban
(8b) bivalrudin
(8c) SSR-182289A
(9) Razaxaban (DPC-906)
(10) 5-chloro-N-[((5S)-2-oxo-3-[4-(3-oxo-4-morpholinyl)phenyl]-1,3-oxazolidin-5-yl)methyl]-2-thiophencarboxamide (BAY-59-7939)
(10a) 1-(4-methoxyphenyl)-7-oxo-6-[4-(2-oxopiperidin-1-yl)phenyl]-4,5,6,7-tetrahydro-1H-pyrazolo[3,4-c]pyridine-3-carboxylic acid amide (INN name: Apixaban),
(11)-(indole-6-carbonyl-D-phenylglycinyl)-4-(1-methyl-piperidin-4-yl)piperazin (LY-517717)
(12) 2-(5-carbamimidoyl-2-hydroxy-phenyl)-N-[3-methyl-4-(pyrrolidin-1-yl-carbonyl)-phenyl]-acetamide
(13) 2-(3-carbamimidoyl-phenyl)-N-[3-methyl-4-(pyrrolidin-1-yl-carbonyl)-phenyl]-isobutyramide
(14) 2-(5-carbamimidoyl-2-hydroxy-phenyl)-N-[4-(pyrrolidin-1-yl-carbonyl)-3-trifluoromethyl-phenyl]-propionamide
(15) 2-(3-carbamimidoyl-phenyl)-N-[3-bromo-4-(pyrrolidin-1-yl-carbonyl)-phenyl]-3-(pyridin-4-yl)-propionamide
(16) N-(5-carbamimidoyl-2-hydroxy-benzyl)-3-methyl-4-(pyrrolidin-1-yl-carbonyl)-benzamide
(17) ethyl 2-(3-carbamimidoyl-phenyl)-2-[3-methyl-4-(pyrrolidin-1-yl-carbonyl)-benzoylamino]-acetate
(18) (1) N-(5-Amidino-2-hydroxy-benzyl)-3-trifluormethyl-4-(3-aminomethyl-1,4,5,6-tetrahydro-cyclopentapyrazol-1-yl)-benzamide
(19) 6) N-[1-(5-Amidino-2-hydroxy-phenyl)-ethyl]-3-trifluormethyl-4-(4,5,6,7-tetrahydro-benzimidazol-1-yl)-benzamide
(20) N-(5-Amidino-2-hydroxy-benzyl)-3-trifluormethyl-4-(3-methyl-1,4,5,6-tetrahydro-cyclopentapyrazol-1-yl)-benzamide
(21) 2-(5-amidino-2-hydroxy-phenyl)-N-[3-trifluoromethyl-4-(pyrrolidin-1-yl-carbonyl phenyl]-3-phenyl-propionamide
(22) 4-hydroxy-3-{[6-chloro-7-(pyrrolidin-1-yl-carbonyl)-quinazolin-4-yl]aminomethyl}-benzamidine
(23) 4-hydroxy-3-{[7-methoxy-6-(pyrrolidin-1-yl-carbonyl)-isoquinolin-1-yl]aminomethyl}-benzamidine
(24) 4-hydroxy-3-{2-phenyl-1-[7-(pyrrolidin-1-yl-carbonyl)-quinazolin-4-ylamino]-ethyl}-benzamidine
(25) 4-hydroxy-3-{[6-methyl-7-(pyrrolidin-1-yl-carbonyl)-quinazolin-4-yl]aminomethyl}-benzamidine
(26) 4-hydroxy-3-{[7-(pyrrolidin-1-yl-carbonyl)-quinazolin-4-yl]aminomethyl}-benzamidine
(27) ethyl 3-(3-amidino-phenyl)-3-{[6-chloro-7-(pyrrolidin-1-yl-carbonyl)-quinazolin-4-yl]amino}-propionate
(28) 3-(3-amidino-phenyl)-3-{[6-chloro-7-(pyrrolidin-1-yl-carbonyl)-quinazolin-4-yl]amino}propionic acid
(29) N-benzoyl-4-hydroxy-3-{[7-(pyrrolidin-1-yl-carbonyl)-quinazolin-4-yl]aminomethyl}-benzamidine
(30) N-hydroxy-4-hydroxy-3-{[6-methyl-7-(pyrrolidin-1-yl-carbonyl)-quinazolin-4-yl]aminomethyl}-benzamidine
(31) N-acetoxymethoxycarbonyl-4-hydroxy-3-{[6-methyl-7-(pyrrolidin-1-yl-carbonyl)-quinazolin-4-yl]aminomethyl}-benzamidine
(32) 1-methyl-2-[N-(4-amidinophenyl)-aminomethyl]-5-[N-(hydroxycarbonylmethyl)-quinoline-8-sulphonylamino]-benzimidazole
(33) (R)-2-(4-amidinophenylaminomethyl)-1-methyl-5-[1-(carboxymethylamino)-1-(pyrrolidinocarbonyl)-ethyl]-benzimidazole
(34) 2-(4-amidinophenylaminomethyl)-1-methyl-5-[1-(carboxymethylaminomethyl)-1-(pyrrolidinocarbonyl)-ethyl]-benzimidazole
(35) (R)-2-[4-(N-phenylcarbonylamidino)-phenylaminomethyl]-1-methyl-5-[1-(n-propyloxycarbonylmethylamino)-1-(pyrrolidinocarbonyl)-ethyl]-benzimidazole
(36) 3-{[6-(N-acetyl-N-cyclopentylamino)-7-methyl-isoquinolin-1-yl]aminomethyl}-4-hydroxy-benzamidine
(37) N-[1-(5-chloro-1H-benzimidazol-2-yl)-ethyl]-3-methyl-4-(2,5-dihydro-pyrrol-1-yl-carbonyl)-benzamide
(38) N-[1-(5-chloro-1H-benzimidazol-2-yl ethyl]-3-ethyl-4-(pyrrolidin-1-yl-carbonyl)-benzamide
(39) N-[1-(5-chloro-1H-benzimidazol-2-yl)-ethyl]-3-chloro-4-(2-aminomethyl-pyrrolidin-1-yl-carbonyl)-benzamide
(40) 3-chloro-N-(5-chloro-1H-benzimidazol-2-yl-methyl)-4-(3-oxo-piperazin-1-yl-carbonyl)-benzamide
(41) N-[1-(5-bromo-1H-benzimidazol-2-yl)-ethyl]-3-methyl-4-(pyrrolidin-1-yl-carbonyl)-benzamide
(42) N-[(5-chloro-1H-benzimidazol-2-yl)-phenyl-methyl]-3-methyl-4-(pyrrolidin-1-yl-carbonyl)-benzamide
(43) N-[1-(5-chloro-1H-benzimidazol-2-yl)-3-methyl-butyl]-3-methyl-4-(pyrrolidin-1-yl-carbonyl)-benzamide
(44) (S)-N-[1-(5-chloro-1H-benzimidazol-2-yl)]ethyl-3-methyl-4-(pyrrolidin-1-yl-1-carbonyl)-benzamide
(45) N-[(1S)-1-(5-chloro-1H-benzimidazol-2-yl)-ethyl]-3-chloro-4-[(2R/S)-2-aminomethyl-pyrrolidin-1-yl-carbonyl)-benzamide
(46) N-[(1S)-1-(5-chloro-1H-benzimidazol-2-yl)-3-methylsulphanyl-propyl]-3-chloro-4-[(2S)-2-(N-tert.-butoxycarbonyl-aminomethyl)-pyrrolidin-1-yl-carbonyl]-benzamide
(47) N-[(1S)-1-(5-chloro-1H-benzimidazol-2-yl)-butyl]-3-chloro-4-[(2S)-2-aminomethyl-pyrrolidin-1-yl-carbonyl]-benzamide
(48) N-[(1S)-1-(5-chloro-1H-benzimidazol-2-yl)-3-methylsulphanyl-propyl]-3-chloro-4-[(2S)-2-aminomethyl-pyrrolidin-1-yl-carbonyl]-benzamide
(49) N-[(1S)-1-(5-chloro-1H-benzimidazol-2-yl)-3-methylsulphinyl-propyl]-3-chloro-4-[(2S)-2-aminomethyl-pyrrolidin-1-yl-carbonyl]-benzamide
(50) N-[(1S)-1-(5-chloro-1H-benzimidazol-2-yl)-3-methylsulphonyl-propyl]-3-chloro-4-[(2S)-2-aminomethyl-pyrrolidin-1-yl-carbonyl]-benzamide
(51) N-[(1S)-5-(benzyloxycarbonylamino)-1-(5-chloro-1H-benzimidazol-2-yl)-pentyl]-3-methyl-4-(pyrrolidin-1-yl-carbonyl)-benzamide
(52) N-[(1S)-1-(5-chloro-1H-benzimidazol-2-yl)-3-phenyl-propyl]-3-methyl-4-(pyrrolidin-1-yl-carbonyl)-benzamide
(53) N-[(1S)-1-(5-chloro-1H-benzimidazol-2-yl)-3-methylsulphanyl-propyl]-3-methyl-4-(pyrrolidin-1-yl-carbonyl)-benzamide
(54) N-[(1S)-3-benzyloxycarbonyl-1-(5-chloro-1H-benzimidazol-2-yl)-propyl]-3-methyl-4-(pyrrolidin-1-yl-carbonyl)-benzamide
(55) N-[(1S)-1-(5-chloro-1H-benzimidazol-2-yl)-3-(pyrrolidin-1-yl-carbonyl)-propyl]-3-methyl-4-(pyrrolidin-1-yl-carbonyl)-benzamide
(56) N-[(1R)-1-(5-chloro-1H-benzimidazol-2-yl)-2-hydroxy-ethyl]-3-methyl-4-(pyrrolidin-1-yl-carbonyl)-benzamide
(57) N-[1-(5-chloro-1H-benzimidazol-2-yl)-2-methoxy-ethyl]-3-methyl-4-(pyrrolidin-1-yl-carbonyl)-benzamide
(58) N-[(1S)-1-(5-chloro-1H-benzimidazol-2-yl)-3-methoxy-propyl]-3-methyl-4-(pyrrolidin-1-yl-carbonyl)-benzamide
(59) N-[(1R)-2-(C-tert.butoxycarbonyl-methyloxy)-1-(5-chloro-1H-benzimidazol-2-yl)-ethyl]-3-methyl-4-(pyrrolidin-1-yl-carbonyl)-benzamide
(60) N-[(1S)-1-(5-chloro-1H-benzimidazol-2-yl)-3-methylsulphinyl-propyl]-3-methyl-4-(pyrrolidin-1-yl-carbonyl)-benzamide
(61) N-[(5-chloro-1H-benzimidazol-2-yl)-phenyl-methyl]-3-methyl-4-(pyrrolidin-1-yl-carbonyl)-benzamide
(62) N-[1-(5-chloro-1H-benzimidazol-2-yl)-phenyl-methyl]-4-(2,5-dihydro-pyrrol-1-yl-carbonyl)-3-methyl-benzamide
(63) N-[(1S)-1-(5-chloro-1H-benzimidazol-2-yl)-3-methylsulphonylamino-propyl]-3-methyl-4-(pyrrolidin-1-yl-carbonyl)-benzamide
(64) N-{(1S)-1-(5-chloro-1H-benzimidazol-2-yl)-3-[3-(2-chloro-ethyl ureido]-propyl}-3-methyl-4-(pyrrolidin-1-yl-carbonyl)-benzamide
(65) N-[(1S)-1-(5-chloro-1H-benzimidazol-2-yl)-butyl]-3-methyl-4-(pyrrolidin-1-yl-carbonyl)-benzamide
(66) 3-bromo-N-[(1S)-1-(5-chloro-1H-benzimidazol-2-yl)-3-methylsulphanyl-propyl]-4-(pyrrolidin-1-yl-carbonyl)-benzamide
(67) 3-chloro-N-[(1S)-1-(5-chloro-1H-benzimidazol-2-yl)-3-(methylsulphanyl)-propyl]-4-(pyrrolidin-1-yl-carbonyl)-benzamide
(68) 3-bromo-N-[(1S)-1-(5-chloro-1H-benzimidazol-2-yl)-3-(methylsulphonyl)-propyl]-4-(pyrrolidin-1-yl-carbonyl)-benzamide
(69) 3-bromo-N-[(1S)-1-(5-chloro-1H-benzimidazol-2-yl)-3-methylsulphinyl-propyl]-4-(pyrrolidin-1-yl-carbonyl)-benzamide
(70) 3-chloro-N-[(1S)-1-(5-chloro-1H-benzimidazol-2-yl)-ethyl]-4-[(2R)-2-(methylsulphonylamino-methyl)-pyrrolidin-1-yl-carbonyl]-benzamide
(71) (1R)-3-bromo-N-[1-(5-chloro-1H-benzimidazol-2-yl)-2-hydroxy-ethyl]-4-(2,5-dihydro-pyrrol-1-yl-carbonyl)-benzamide
(72) (1R)-3-methyl-N-[1-(5-chloro-1H-benzimidazol-2-yl)-2-methoxy-ethyl]-4-(2,5-dihydro-pyrrol-1-yl-carbonyl)-benzamide
(73) (1R)-3-chloro-N-[1-(5-chloro-1H-benzimidazol-2-yl)-2-hydroxy-ethyl]-4-(2,5-dihydro-pyrrol-1-yl-carbonyl)-benzamide
(74) N-{(1S)-1-(5-chloro-1H-benzimidazol-2-yl)-3-[(3R,S)-3-dimethylamino-pyrrolidin-1-yl]-carbonyl-propyl}-3-methyl-4-(pyrrolidin-1-yl-carbonyl)-benzamide
(75) N-{(1S)-1-(5-chloro-1H-benzimidazol-2-yl)-3-[(2R)-2-hydroxymethyl-pyrrolidin-1-yl-carbonyl]-propyl}-3-methyl-4-(pyrrolidin-1-yl-carbonyl)-benzamide
(76) N-{(1S)-1-(5-chloro-1H-benzimidazol-2-yl)-3-[(2S)-2-hydroxymethyl-pyrrolidin-1-yl-carbonyl]-propyl}-3-methyl-4-(pyrrolidin-1-yl-carbonyl)-benzamide
(77) N-[(1S)-1-(5-chloro-1H-benzimidazol-2-yl)-3-(2-methyl-2,6-diaza-spiro[3.4]oct-6-yl-carbonyl)-propyl]-3-methyl-4-(pyrrolidin-1-yl-carbonyl)-benzamide
(78) N-{(1S)-3-[(1R)-2-(aminocarbonyl)-pyrrolidin-1-yl-carbonyl]-1-(5-chloro-1H-benzimidazol-2-yl)-propyl}-3-methyl-4-(pyrrolidin-1-yl-carbonyl)-benzamide
(79) N-{(1S)-1-(5-chloro-1H-benzimidazol-2-yl)-3-[(2R)-2-tert.butoxycarbonyl-aminomethyl-pyrrolidin-1-yl-carbonyl]-propyl}-3-methyl-4-(pyrrolidin-1-yl-carbonyl)-benzamide
(80) N-{(1S)-1-(5-chloro-1H-benzimidazol-2-yl)-3-[(3R,S)-hydroxymethyl-pyrrolidin-1-yl)-carbonyl]-propyl}-3-methyl-4-(pyrrolidin-1-yl-carbonyl)-benzamide
(81) N-[(1S)-1-(5-chloro-1H-benzimidazol-2-yl)-3-(1,1-dioxo-1-thiomorpholine-4-yl-carbonyl]-propyl-3-methyl-4-(pyrrolidin-1-yl-carbonyl)-benzamide
(82) N-[(1S)-1-(5-chloro-1H-benzimidazol-2-yl)-3-[(4-methyl-3-oxo-piperazin-1-yl-carbonyl)-propyl]-3-methyl-4-(pyrrolidin-1-yl-carbonyl)-benzamide
(83) N-[(1R)-1-(5-chloro-1H-benzimidazol-2-yl)-2-methoxy-ethyl]-3-methyl-4-(pyrrolidin-1-yl-carbonyl)-benzamide
(84) 3-chloro-N-[(1R)-1-(5-chloro-1H-benzimidazol-2-yl)-2-methoxy-ethyl]-4-(2,5-dihydro-pyrrol-1-yl-carbonyl)-benzamide
(85) 3-bromo-N-[(1R)-1-(5-chloro-1H-benzimidazol-2-yl)-2-methoxy-ethyl]-4-(pyrrolidin-1-yl-carbonyl)-benzamide
(86) 3-bromo-N-[(1R)-1-(5-chloro-1H-benzimidazol-2-yl)-2-methoxy-ethyl]-4-(2,5-dihydro-pyrrol-1-yl-carbonyl)-benzamide
(87) 3-methyl-N-[(1R)-1-(5-chloro-1H-benzimidazol-2-yl)-2-hydroxy-ethyl]-4-(2,5-dihydro-pyrrol-1-yl-carbonyl)-benzamide
(88) N-{(1S)-1-(5-chloro-1H-benzimidazol-2-yl)-3-[(2S)-2-aminomethyl-pyrrolidin-1-yl-carbonyl]-propyl}-3-methyl-4-(pyrrolidin-1-yl-carbonyl)-benzamide
(89) N-{(1S)-1-(5-chloro-1H-benzimidazol-2-yl)-3-[(2R)-2-aminomethyl-pyrrolidin-1-yl-carbonyl]-propyl}-3-methyl-4-(pyrrolidin-1-yl-carbonyl)-benzamide
(90) 3-chloro-N-[(1R,S)-1-(5-chloro-1H-benzimidazol-2-yl)-ethyl]-4-[(2R)-2-methoxymethyl-pyrrolidin-1-yl-carbonyl]-benzamide
(91) 3-chloro-N-[1-(5-chloro-1H-benzimidazol-2-yl)-ethyl]-4-(3,4,5,6-tetrahydro-2H-[2,3]-bipyridinyl-1-yl-carbonyl)-benzamide
(92) N-[(1R)-1-(5-chloro-1H-benzimidazol-2-yl)-2-methoxy-ethyl]-4-(pyrrolidin-1-yl-carbonyl)-3-trifluoromethyl-benzamide
(93) N-[(1S)-1,3-bis-(5-chloro-1H-benzimidazol-2-yl)-propyl]-3-methyl-4-(pyrrolidin-1-yl-carbonyl)-benzamide
(94) 3-chloro-N-[(1S)-1-(5-chloro-1H-benzimidazol-2-yl)-ethyl]-4-[(2R/S)-2-dimethylaminomethyl-pyrrolidin-1-yl-carbonyl]-benzamide
(95) N-[(1S)-1-(5-chloro-1H-benzimidazol-2-yl)-3-methanesulphonylamino-propyl]-4-(2,5-dihydro-pyrrol-1-yl-carbonyl)-3-methyl-benzamide
(96) N-[(1S)-1-(5-chloro-1H-benzimidazol-2-yl)-butyl]-4-(2,5-dihydro-pyrrol-1-yl-carbonyl)-3-methyl-benzamide
(97) 3-chloro-N-[(1S)-1-(5-chloro-11H-benzimidazol-2-yl)-butyl]-4-(2,5-dihydro-pyrrol-1-yl-carbonyl)-benzamide
(98) 3-bromo-N-[(1S)-1-(5-chloro-1H-benzimidazol-2-yl)-butyl]-4-(2,5-dihydro-pyrrol-1-yl-carbonyl)-benzamide
(99) 4-(N-acetyl-N-cyclopentyl-amino)-N-[(1S)-1-(5-chloro-1H-benzimidazol-2-yl)-2-methylsulphanyl-ethyl]-3-methyl-benzamide
(100) 3-chloro-N-[(1S)-1-(5-chloro-1H-benzimidazol-2-yl)-ethyl]-4-[(2R)-2-(pyrrolidin-1-yl-methyl)-pyrrolidin-1-yl-carbonyl]-benzamide
(101) 3-bromo-N-[(1R)-1-(5-bromo-1H-benzimidazol-2-yl)-2-methoxy-ethyl]-4-(2,5-dihydro-pyrrol-1-yl-carbonyl)-benzamide
(102) 3-bromo-N-[(1R)-1-(5-chloro-1H-benzimidazol-2-yl)-2-ethoxy-ethyl]-4-(2,5-dihydro-pyrrol-1-yl-carbonyl)-benzamide
(103) N-[(1R)-2-allyloxy-1-(5-chloro-1H-benzimidazol-2-yl)-ethyl]-4-(2,5-dihydro-pyrrol-1-yl-carbonyl)-3-methyl-benzamide
(104) 3-bromo-N-[(1R)-1-(5-chloro-1H-benzimidazol-2-yl)-2-prop-2-ynyloxy-ethyl]-4-(2,5-dihydro-pyrrol-1-yl-carbonyl)-benzamide
(105) N-[(1S)-1-(5-chloro-1H-benzimidazol-2-yl)-3-(1H-tetrazol-5-yl)-propyl]-3-methyl-4-(pyrrolidin-1-yl-carbonyl)-benzamide
(106) N-[(1R)-1-(5-chloro-1H-benzimidazol-2-yl)-2-hydroxy-ethyl]-4-(2,5-dihydro-pyrrol-1-yl-carbonyl)-3-trifluoromethyl-benzamide
(107) 3-chloro-N-[(1R)-1-(5-bromo-1H-benzimidazol-2-yl)-2-hydroxy-ethyl]-4-(2,5-dihydro-pyrrol-1-yl-carbonyl)-benzamide
(108) 3-bromo-N-[(1R)-1-(5-bromo-1H-benzimidazol-2-yl)-2-hydroxy-ethyl]-4-(pyrrolidin-1-yl-carbonyl)-benzamide
(109) 3-methyl-N-[(1R)-1-(5-bromo-1H-benzimidazol-2-yl)-2-hydroxy-ethyl]-4-(pyrrolidin-1-yl-carbonyl)-benzamide
(110) 6-chloro-4-[1-(5-chloro-1H-benzimidazol-2-yl)-ethylamino]-7-(2-aminomethyl-pyrrolidin-1-yl-carbonyl)-quinazoline
(111) 6-chloro-4-[1-(S)-(5-chloro-1H-benzimidazol-2-yl)-ethylamino]-7-(2,5-dihydropyrrol-1-yl-carbonyl)-quinazoline
(112) 6-chloro-4-[1-(S)-(5-chloro-1H-benzimidazol-2-yl)-ethylamino]-7-(pyrrolidin-1-yl-carbonyl)-quinazoline
(113) 4-[1-(5-chloro-1H-benzimidazol-2-yl)-3-methylsulphanyl-propylamino]-6-methyl-7-(pyrrolidin-1-yl-carbonyl)-quinoline
(114) 4-[1-(5-chloro-1H-benzimidazol-2-yl)-ethylamino]-6-methyl-7-(pyrrolidin-1-yl-carbonyl)-quinoline
(115) 4-[1-(5-chloro-1H-benzimidazol-2-yl)-ethylamino]-6-methyl-7-(3-oxo-piperazin-1-yl-carbonyl)-quinoline
(116) 4-[(1R/S)-1-(5-chloro-1H-benzimidazol-2-yl)-ethylamino]-6-methyl-7-[(2R)-2-aminomethyl-pyrrolidin-1-yl-carbonyl]-quinoline
(117) 4-[1-(5-chloro-1H-benzimidazol-2-yl)-3-methylsulphanyl-propylamino]-6-methyl-7-(3-oxo-piperazin-1-yl-carbonyl)-quinoline
(118) 4-[1-(5-chloro-1H-benzimidazol-2-yl)-3-methanesulphonyl-propylamino]-6-methyl-7-(pyrrolidin-1-yl-carbonyl)-quinoline
(119) 6-chloro-4-[(1S)-1-(5-chloro-1H-benzimidazol-2-yl)-ethylamino]-7-[(2R)-2-aminomethyl-pyrrolidin-1-yl-carbonyl]-quinazoline
(120) 6-chloro-4-[(1S)-1-(5-chloro-1H-benzimidazol-2-yl)-2-hydroxy-ethylamino]-7-(2,5-dihydropyrrol-1-yl-carbonyl)-quinazoline
(121) 6-chloro-4-[(1S)-1-(5-chloro-1H-benzimidazol-2-yl)-2-hydroxy-ethylamino]-7-[(2R)-2-aminomethyl-pyrrolidin-1-yl-carbonyl]-quinazoline
(122) 6-chloro-4-[1-(5-chloro-1H-benzimidazol-2-yl)-3-hydroxycarbonylpropylamino]-7-(pyrrolidin-1-yl-carbonyl)-quinazoline
(123) 6-chloro-4-[1-(5-chloro-1H-benzimidazol-2-yl)-3-benzyloxycarbonylpropyl-amino]-7-(pyrrolidin-1-yl-carbonyl)-quinazoline
(124) 6-chloro-4-[(1S)-1-(5-chloro-1H-benzimidazol-2-yl)-3-methylsulphanyl-propylamino]-7-[(2R)-2-tert.-butyloxycarbonyl-aminomethyl-pyrrolidin-1-yl-carbonyl]-quinazoline
(125) 6-chloro-4-[(1S)-1-(5-chloro-1H-benzimidazol-2-yl)-3-methylsulphanyl-propylamino]-7-(pyrrolidin-1-yl-carbonyl)-quinazoline
(126) 6-chloro-4-[(1S)-1-(5-chloro-1H-benzimidazol-2-yl)-3-methoxy-propylamino]-7-(2,5-dihydropyrrol-1-yl-carbonyl)-quinazoline
(127) 6-chloro-4-[(1S)-1-(5-chloro-1H-benzimidazol-2-yl)-3-methanesulphonyl-propylamino]-7-(pyrrolidin-1-yl-carbonyl)-quinazoline
(128) 6-chloro-4-[(1S)-1-(5-chloro-1H-benzimidazol-2-yl)-3-methylsulphanyl-propylamino]-7-[(2R)-2-aminomethyl-pyrrolidin-1-yl-carbonyl]-quinazoline
(129) 6-chloro-4-[(1S)-1-(5-chloro-1H-benzimidazol-2-yl)-3-methanesulphinyl-propylamino]-7-(pyrrolidin-1-yl-carbonyl)-quinazoline
(130) 6-chloro-4-[(1S)-1-(5-chloro-1H-benzimidazol-2-yl)-3-benzyloxycarbonyl-propylamino]-7-(pyrrolidin-1-yl-carbonyl)-quinazoline
(131) 6-chloro-4-[(1S)-1-(5-chloro-1H-benzimidazol-2-yl)-2-hydroxy-ethylamino]-7-(piperazin-3-on-1-yl-carbonyl)-quinazoline
(132) 6-chloro-4-[(1S)-1-(5-chloro-1H-benzimidazol-2-yl)-3-hydroxycarbonylpropyl-amino]-7-[(2S)-2-aminomethyl-pyrrolidin-1-yl-carbonyl]-quinazoline
(133) 6-chloro-4-[(1S)-1-(5-chloro-1H-benzimidazol-2-yl)-3-methanesulphonyl-propylamino]-7-[(2R)-2-tert.-butyloxycarbonyl-aminomethyl-pyrrolidin-1-yl-carbonyl]-quinazoline
(134) 6-chloro-4-[(1S)-1-(5-chloro-1H-benzimidazol-2-yl)-3-methanesulphonyl-propylamino]-7-[(2R)-2-aminomethyl-pyrrolidin-1-yl-carbonyl]-quinazoline
(135) 6-chloro-4-[(1S)-1-(5-chloro-1H-benzimidazol-2-yl)-ethylamino]-7-(thiazolidin-3-yl-carbonyl)-quinazoline
(136) 6-chloro-4-[(1S)-1-(5-chloro-1H-benzimidazol-2-yl)-3-ethoxycarbonylpropyl-amino]-7-(pyrrolidin-1-yl-carbonyl)-quinazoline
(137) 4-[(1S)-1-(5-chloro-1H-benzimidazol-2-yl)-ethylamino]-6-methyl-7-(pyrrolidin-1-yl-carbonyl)-quinazoline
(138) 4-[(1S)-1-(5-chloro-1H-benzimidazol-2-yl)-ethylamino]-6-methyl-7-(pyrrolidin-1-yl-carbonyl)-quinazoline
(139) 6-chloro-4-[(1S)-1-(5-chloro-1H-benzimidazol-2-yl)-3-methanesulphinyl-propylamino]-7-[(2R)-2-aminomethyl-pyrrolidin-1-yl-carbonyl]-quinazoline
(140) 4-[(1S)-1-(5-chloro-1H-benzimidazol-2-yl)-3-methylsulphanyl-propylamino]-6-methyl-7-(2,5-dihydropyrrol-1-yl-carbonyl)-quinazoline
(141) 6-bromo-4-[(1S)-1-(5-chloro-1H-benzimidazol-2-yl)-ethylamino]-7-(2,5-dihydropyrrol-1-yl-carbonyl)-quinazoline
(142) 6-chloro-4-[(1S)-1-(5-chloro-1H-benzimidazol-2-yl)-3-ethoxycarbonylpropyl-amino]-7-(2,5-dihydropyrrol-1-yl-carbonyl)-quinazoline
(143) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(144) 6-chloro-4-[(1S)-1-(5-chloro-1H-benzimidazol-2-yl)-butylamino]-7-(2,5-dihydropyrrol-1-yl-carbonyl)-quinazoline
(145) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(146) 6-chloro-4-[(1S)-1-(5-chloro-1H-benzimidazol-2-yl)-2-methoxy-ethylamino]-7-(2,5-dihydropyrrol-1-yl-carbonyl)-quinazoline
(147) 6-chloro-4-[1-(5-chloro-1H-benzimidazol-2-yl)-3-diethylaminocarbonyl-propyl-amino]-7-(pyrrolidin-1-yl-carbonyl)-quinazoline
(148) 6-chloro-4-[1-(5-chloro-1H-benzimidazol-2-yl)-3-[N-methyl-N-piperidin-4-yl-amino]-carbonyl-propyl-amino]-7-(pyrrolidin-1-yl-carbonyl)-quinazoline
(149) 6-chloro-4-[1-(5-chloro-1H-benzimidazol-2-yl)-3-[4-methyl-piperazin-1-yl]-carbonyl-propyl-amino]-7-(pyrrolidin-1-yl-carbonyl)-quinazoline
(150) 6-chloro-4-[1-(5-chloro-1H-benzimidazol-2-yl)-3-(C-piperidin-4-yl-methylamino)-carbonyl-propyl-amino]-7-(pyrrolidin-1-yl-carbonyl)-quinazoline
(151) 6-chloro-4-[1-(5-chloro-1H-benzimidazol-2-yl)-3-(N-benzyl-N-methyl-amino)-carbonyl-propyl-amino]-7-(pyrrolidin-1-yl-carbonyl quinazoline
(152) 4-[(1S)-1-(5-chloro-1H-benzimidazol-2-yl)-3-allyloxycarbonylpropyl-amino]-6-methyl-7-(2,5-dihydropyrrol-1-yl-carbonyl)-quinazoline
(153) 6-bromo-4-[(1S)-1-(5-chloro-1H-benzimidazol-2-yl)-3-allyloxycarbonylpropyl-amino]-7-(2,5-dihydropyrrol-1-yl-carbonyl quinazoline
(154) 6-chloro-4-[(1S)-1-(5-chloro-1H-benzimidazol-2-yl)-2-methoxy-ethylamino]-7-(pyrrolidin-1-yl-carbonyl)-quinazoline
(155) 6-chloro-4-[(1S)-1-(5-chloro-1H-benzimidazol-2-yl)-3-methanesulphonyl-propylamino]-1-oxy-7-[(2R)-2-aminomethyl-pyrrolidin-1-yl-carbonyl]-quinazoline
(156) 6-chloro-4-[(1S)-1-(5-chloro-1H-benzimidazol-2-yl)-ethylamino]-7-[(2S)-2-(pyrrolidin-1-yl-methyl)-pyrrolidin-1-yl-carbonyl]-quinazoline
(157) 6-chloro-4-[(1S)-1-(5-chloro-1H-benzimidazol-2-yl)-ethylamino]-7-[(2R/S)-2-aminomethyl-thiazolidinyl-carbonyl]-quinazoline
(158) 6-chloro-4-[(1S)-1-(5-chloro-1H-benzimidazol-2-yl)-3-methanesulphonyl-propylamino]-7-[(2R)-2-(methanesulphonyl-aminomethyl)-pyrrolidin-1-yl-carbonyl]-quinazoline
(159) 6-chloro-4-{1-(5-chloro-1H-benzimidazol-2-yl)-3-[(1,2,3,4-tetrahydroiso-quinolin-1-yl)-carbonyl-propyl-amino]}-7-(pyrrolidin-1-yl-carbonyl)-quinazoline
(160) 6-chloro-4-[1-(5-chloro-1H-benzimidazol-2-yl)-3-(benzylamino-carbonyl)-propyl-amino]-7-(pyrrolidin-1-yl-carbonyl)-quinazoline
(161) 6-chloro-4-{1-(5-chloro-1H-benzimidazol-2-yl)-3-[(N-methyl-N-phenethyl-amino-carbonyl)-propyl-amino]}-7-(pyrrolidin-1-yl-carbonyl)-quinazoline
(162) 6-chloro-4-[1-(5-chloro-1H-benzimidazol-2-yl)-3-(hydroxyethylamino-carbonyl)-propyl-amino]-7-(pyrrolidin-1-yl-carbonyl)-quinazoline
(163) 6-chloro-4-{1-(5-chloro-1H-benzimidazol-2-yl)-3-[(C-pyridin-3-yl-methylamino-carbonyl)-propyl-amino]}-7-(pyrrolidin-1-yl-carbonyl)-quinazoline
(164) 6-chloro-4-{1-(5-chloro-1H-benzimidazol-2-yl)-3-[(1-oxa-3,8-diaza-spiro[4.5]decan-2-on-8-yl carbonyl]-propyl-amino}-7-(pyrrolidin-1-yl-carbonyl)-quinazoline
(165) 6-chloro-4-[1-(5-chloro-1H-benzimidazol-2-yl)-3-(morpholin-4-yl-carbonyl)-propyl-amino]-7-(pyrrolidin-1-yl-carbonyl quinazoline
(166) 6-chloro-4-[1-(5-chloro-1H-benzimidazol-2-yl)-3-(C-cyclohexyl-methylamino-carbonyl)-propyl-amino]-7-(pyrrolidin-1-yl-carbonyl)-quinazoline
(167) 6-chloro-4-[1-(5-chloro-1H-benzimidazol-2-yl)-3-(methoxyethylamino-carbonyl)-propyl-amino]-7-(pyrrolidin-1-yl-carbonyl)-quinazoline
(168) 6-chloro-4-[1-(5-chloro-1H-benzimidazol-2-yl)-3-(dimethylaminoethyl-amino-carbonyl)-propyl-amino]-7-(pyrrolidin-1-yl-carbonyl)-quinazoline
(169) 6-chloro-4-[1-(5-chloro-1H-benzimidazol-2-yl)-3-(cyclopropylamino-carbonyl)-propyl-amino]-7-(pyrrolidin-1-yl-carbonyl)-quinazoline
(170) 6-chloro-4-{(1R/S)-1-(5-chloro-1H-benzimidazol-2-yl)-3-[C-(2R/S)-tetrahydrofuran-2-yl-methylamino-carbonyl)-propyl-amino]}-7-(pyrrolidin-1-yl-carbonyl)-quinazoline
(171) 6-chloro-4-[1-(5-chloro-1H-benzimidazol-2-yl)-3-(dimethylaminopropylamino-carbonyl)-propyl-amino]-7-(pyrrolidin-1-yl-carbonyl)-quinazoline
(172) 6-chloro-4-[1-(5-chloro-1H-benzimidazol-2-yl)-3-(aminoethylamino-carbonyl)-propyl-amino]-7-(pyrrolidin-1-yl-carbonyl)-quinazoline
(173) 6-chloro-4-[1-(5-chloro-1H-benzimidazol-2-yl)-3-(2,2,2-trifluoroethylamino-carbonyl)-propyl-amino]-7-(pyrrolidin-1-yl-carbonyl)-quinazoline
(174) 6-chloro-4-{1-(5-chloro-1H-benzimidazol-2-yl)-3-[N-(2-dimethylamino-ethyl)-N-methyl-amino-carbonyl]-propyl-amino}-7-(pyrrolidin-1-yl-carbonyl)-quinazoline
(175) 6-chloro-4-[1-(5-chloro-1H-benzimidazol-2-yl)-3-(N-piperidin-2-yl-aminocarbonyl)-propyl-amino]-7-(pyrrolidin-1-yl-carbonyl)-quinazoline
(176) 6-chloro-4-{1-(5-chloro-1H-benzimidazol-2-yl)-3-[C-(tetrahydropyran-4-yl)-methylamino-carbonyl]-propyl-amino}-7-(pyrrolidin-1-yl-carbonyl)-quinazoline
(177) 6-chloro-4-[1-(5-chloro-1H-benzimidazol-2-yl)-3-(4-hydroxypiperidin-1-yl-carbonyl)-propyl-amino]-7-(pyrrolidin-1-yl-carbonyl)-quinazoline
(178) 6-chloro-4-{1-(5-chloro-1H-benzimidazol-2-yl)-3-[C-(pyridin-4-yl)-methylamino-carbonyl]-propyl-amino}-7-(pyrrolidin-1-yl-carbonyl)-quinazoline
(179) 6-chloro-4-[1-(5-chloro-1H-benzimidazol-2-yl)-3-(N-methylaminocarbonylmethyl-N-methyl-amino-carbonyl)-propyl-amino]-7-(pyrrolidin-1-yl-carbonyl)-quinazoline
(180) 6-chloro-4-{1-(5-chloro-1H-benzimidazol-2-yl)-3-[N-(2-(1H)-imidazol-4-yl)-ethyl)-N-methyl-amino-carbonyl]-propyl-amino}-7-(pyrrolidin-1-yl-carbonyl)-quinazoline
(181) 6-chloro-4-[1-(5-chloro-1H-benzimidazol-2-yl)-3-(1-thiazolidin-3-yl-carbonyl)-propyl-amino]-7-(pyrrolidin-1-yl-carbonyl)-quinazoline
(182) 6-chloro-4-[1-(5-chloro-1H-benzimidazol-2-yl)-3-(N-cyclopropyl-N-methyl-amino-carbonyl)-propyl-amino]-7-(pyrrolidin-1-yl-carbonyl quinazoline
(183) 6-chloro-4-[1-(5-chloro-1H-benzimidazol-2-yl)-3-(N-cyclopropylmethyl-N-methyl-amino-carbonyl)-propyl-amino]-7-(pyrrolidin-1-yl-carbonyl)-quinazoline
(184) 6-chloro-4-[1-(5-chloro-1H-benzimidazol-2-yl)-3-(cyclopentylamino-carbonyl)-propyl-amino]-7-(pyrrolidin-1-yl-carbonyl)-quinazoline
(185) 6-chloro-4-[1-(5-chloro-1H-benzimidazol-2-yl)-3-(N-piperidin-4-yl-aminocarbonyl)-propyl-amino]-7-(pyrrolidin-1-yl-carbonyl)-quinazoline
(186) 6-chloro-4-{1-(5-chloro-1H-benzimidazol-2-yl)-3-[C-(pyridin-2-yl)-methylamino-carbonyl]-propyl-amino}-7-(pyrrolidin-1-yl-carbonyl)-quinazoline
(187) 6-chloro-4-[(1S)-1-(5-chloro-1H-benzimidazol-2-yl)-3-hydroxycarbonyl-propylamino]-7-(pyrrolidin-1-yl-carbonyl)-quinazoline
(188) 6-chloro-4-[(1S)-1-(5-chloro-1H-benzimidazol-2-yl)-ethylamino]-7-(5,6,7,8-tetrahydro-[1,2,4]triazolo[4,3a]pyridin-4-yl)-quinazoline
(189) 6-chloro-4-[(1S)-1-(5-chloro-1H-benzimidazol-2-yl)-3-(1,1-dioxo-isothiazolidin-2-yl)-propyl-amino]-7-(2,5-dihydropyrrol-1-yl-carbonyl)-quinazoline
(190) 6-chloro-4-[(1S)-1-(5-chloro-1H-benzimidazol-2-yl)-3-methanesulphonylamino-propyl-amino]-7-(2,5-dihydropyrrol-1-yl-carbonyl)-quinazoline
(191) 4-[(1S)-1-(5-chloro-1H-benzimidazol-2-yl)-3-(methylsulphanyl)-propylamino]-6-methoxy-7-(2,5-dihydropyrrol-1-yl-carbonyl)-quinazoline
(192) 4-[(1S)-1-(5-chloro-1H-benzimidazol-2-yl)-2-methoxy-ethylamino]-6-methoxy-7-(2,5-dihydropyrrol-1-yl-carbonyl)-quinazoline
(193) 6-chloro-4-[(1S)-1-(5-chloro-1H-benzimidazol-2-yl)-2-methoxy-ethylamino]-7-(thiazolidinyl-carbonyl)-quinazoline
(194) 4-[(1S)-1-(5-chloro-1H-benzimidazol-2-yl)-2-methoxy-ethylamino]-6-methyl-7-(2,5-dihydropyrrol-1-yl-carbonyl)-quinazoline
(195) 4-[(1S)-1-(5-chloro-1H-benzimidazol-2-yl)-2-methoxy-ethylamino]-6-methyl-7-(thiazolidinyl-carbonyl)-quinazoline
(196) 6-bromo-4-[(1S)-1-(5-chloro-1H-benzimidazol-2-yl)-2-methoxy-ethylamino]-7-(2,5-dihydropyrrol-1-yl-carbonyl)-quinazoline
(197) 6-bromo-4-[(1S)-1-(5-chloro-1H-benzimidazol-2-yl)-2-methoxy-ethylamino]-7-(thiazolidinyl-carbonyl)-quinazoline
(198) 6-chloro-4-[(1S)-1-(5-chloro-1H-benzimidazol-2-yl)-ethylamino]-7-(6,7,8,9-tetrahydro-[1,2,4]triazolo[4,3-a]pyridin-4-yl)-quinazoline
(199) 6-chloro-4-{1-(5-chloro-1H-benzimidazol-2-yl)-3-[2-(pyridin-4-yl-amino)-ethylamino-carbonyl]-propylamino}-7-(pyrrolidin-1-yl-carbonyl quinazoline
(200) 4-[(1S)-1-(5-bromo-1H-benzimidazol-2-yl)-2-methoxy-ethylamino]-6-chloro-7-(2,5-dihydropyrrolyl-carbonyl)-quinazoline and
(201) 4-[(1S)-1-(5-bromo-1H-benzimidazol-2-yl)-ethylamino]-6-chloro-7-(2,5-dihydropyrrolyl-carbonyl)-quinazoline,
their stereoisomers such as enantiomers and diastereomers, mixtures of stereoisomers such as racemates, prodrugs, pharmacologically acceptable salts, solvates, e.g. hydrates, and physical modifications thereof, e.g. polymorphs.

3. The method of claim 1 or 2, wherein the indication is selected from
(a) treatment or prevention of acute myocardial infarction, prevention of myocardial reinfarction,
(b) treatment or prevention of myocardial ischemia (angina pectoris, ischemic heart diseases, chest pain of ischemic etiology), of coronary heart disease or of acute coronary syndromes, secondary prevention of coronary artery disease, treatment and prevention of recurrent ischemic events after acute myocardial infarction, prevention of left ventricular thrombus formation following anterior myocardial infarction,
(c) treatment or prevention of TIA (transient ischemic attacks, or acute cerebrovascular syndromes), of ischemic stroke or prevention of secondary ischemic stroke,
(d) treatment and prevention of complications of (chronic) atrial fibrillation, e.g. stroke prevention in atrial fibrillation,
(d) reduction of the risk for cardiovascular death,
(e) treatment or prevention of ischemic peripheral circulatory disorders, of peripheral vascular disease or of peripheral microcirculation disorders (e.g. Raynaud's disease, tinnitus or sudden loss of hearing),
(f) treatment or prevention of pulmonary hypertension or of pulmonary embolism,
(g) treatment or prevention of thromboembolism, acute treatment and extended secondary prevention of deep vein thrombosis (DVT), prevention of venous thromboembolism after major orthopaedic surgery (e.g. hip or knee replacement),
(h) arterial thrombosis of any vessel, peripheral arterial occlusion, retinal vascular accident, catheter thrombotic occlusion or reocclusion, disseminated intravascular coagulation,
(i) prevention of thromboembolic disorders or complications by endovascular procedures, intra-arterial or intravenous lines, implantation of devices, particularly those exposed to the blood flow, such as stents, prosthetic heart valves, filters, etc, whereby this risk of thrombus formation is reduced by the method of the invention,
(k) prevention of stenosis in vascular grafts, e.g. synthetic vascular grafts, prevention of vascular stent stenosis, prevention of coronary stent stenosis, carotid stent stenosis or peripheral stent stenosis, prevention of stenosis in synthetic grafts used in patients with haemodialysis, prevention of shunt stenosis, prevention of restenosis after angioplasty (e.g. balloon angioplasty, PT(C)A), preventing reocclusions after bypass operations,
in a person in need thereof.

4. The method of any of claims 1 to 3, wherein additionally a platelet aggregation inhibitor selected from acetylsalicalic acid (ASA), clopidogrel, ticlopidine and the pharmaceutically acceptable salts thereof is administered.

5. A pharmaceutical composition comprising a therapeutically effective amount of DIP or a pharmaceutically acceptable salt thereof, and a therapeutically effective amount of an antithrombotic selected from direct thrombin inhibitors, factor Xa inhibitors and combined thrombin/factor Xa inhibitors, optionally together with one or more excipients or carriers.

6. The pharmaceutical composition of claim 5, wherein the antithrombotic is selected from compounds (1) to (201) mentioned in claim 2.

7. The pharmaceutical composition of claim 5 or 6, comprising additionally a therapeutically effective amount of a platelet aggregation inhibitor selected from ASA, clopidogrel and ticlopidine and the pharmaceutically acceptable salts thereof.

8. Use of dipyridamole or a pharmaceutically acceptable salt thereof in combination with an antithrombotic selected from direct thrombin inhibitors, factor Xa inhibitors and combined thrombin/factor Xa inhibitors for the manufacture of a pharmaceutical composition for the treatment of the human or non-human animal body for treating and/or preventing an indication selected from thromboembolic disorders.

9. The use of claim 8, wherein the antithrombotic is selected from compounds (1) to (201) mentioned in claim 2.

10. The use of claim 8 or 9, wherein the indication is selected from the indications mentioned in claim 3.

11. The use of claims 8, 9 or 10, wherein the pharmaceutical composition additionally comprises a platelet aggregation inhibitor selected from ASA, clopidogrel and ticlopidine and the pharmaceutically acceptable salts thereof.
